(19) United States
(12) Patent Application Publication
(10) Pub. No.: US 2026/0290572 A1
(43) Pub. Date: Sep. 24, 2026

(54) SYSTEM

(71) Applicant: SoftBank Group Corp., Tokyo (JP)

(72) Inventor: Kotaro HOSOYAMADA, Tokyo (JP)

(73) Assignee: SoftBank Group Corp., Tokyo (JP)

(21) Appl. No.: 19/567,329

(22) Filed: Mar. 16, 2026

(30) Foreign Application Priority Data
Mar. 19, 2025 (JP) 2025-045112

(51) Int. Cl. G16H 40/63; G06N 5/04

(52) U.S. Cl. CPC G16H 40/63; G06N 5/04

(57) ABSTRACT

A system includes a processor that is configured to perform a learning process by using a prompt to instruct a generative AI model to learn body capability information and motion information, acquire health condition information and motion information of a subject by using at least one of a sensor and a camera, and perform a generation process by using a prompt to instruct the generative AI model to generate an optimal motion pattern, the optimal motion pattern being selected from learned data such that the subject can exhibit a maximum capability with a reduced risk of injury, and being used for presenting a motion of the subject as an image of a three-dimensional model.

### CROSS-REFERENCE TO RELATED APPLICATION

This application is based on and claims priority under 35 USC 119 from Japanese Patent Application No. 2025-045112 filed on Mar. 19, 2025, the disclosure of which is incorporated by reference herein.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

Conventional sports training systems and motion analysis tools mainly focus on recording and replaying an athlete's motion, or on providing generic coaching instructions based on average motion models. Such systems do not sufficiently take into account an individual subject's physical capabilities, medical limitations, or current health condition, and therefore cannot reliably derive an optimal motion pattern for that particular subject. As a result, a subject may fail to fully exhibit the subject's available capability, may continue to use a suboptimal form, and may be exposed to an elevated risk of injury. In addition, existing systems that utilize AI often rely on fixed, pre-trained models and do not effectively employ prompts to control a generative AI model for both learning and generating motion patterns tailored to the subject. Furthermore, even when an improved motion is analytically derived, the motion is not always presented in an intuitive, three-dimensional visual form that allows the subject to easily understand and practice the recommended motion. Therefore, there is a need for a system that uses a generative AI model, controlled by prompts, to learn motion and body capability information, that acquires health condition and motion information of the subject, and that generates and presents an optimal motion pattern as a three-dimensional model image so that the subject can maximize performance while reducing injury risk.

### SUMMARY

To solve the above-described problems, according to one aspect of the present invention, there is provided a system comprising a processor, wherein the processor is configured to perform a learning process by using a prompt to instruct a generative AI model to learn body capability information and motion information, to acquire health condition information and motion information of a subject by using at least one of a sensor and a camera, and to perform a generation process by using a prompt to instruct the generative AI model to generate an optimal motion pattern. In this system, the optimal motion pattern is generated based on learned data such that the subject can exhibit a maximum capability with a reduced risk of injury, and the optimal motion pattern is used for presenting a motion of the subject as an image of a three-dimensional model. In a preferred embodiment, the processor is configured to use a prompt that instructs the generative AI model to read motion data of professional athletes as reference data in the learning process, so that expert-level motion characteristics are reflected in the learned model. Furthermore, the processor is configured to construct the three-dimensional model based on the optimal motion pattern generated by the generative AI model, thereby enabling the subject to visually recognize and practice a motion pattern that allows the subject to exhibit all of the subject's available capability while suppressing injury risk.

The term “system” refers to a combination of hardware and software components including at least one processor, memory, input/output interfaces, and any associated devices or programs, configured to execute the processes described in the claims.

The term “processor” refers to any hardware component or combination of components capable of executing instructions, including but not limited to a central processing unit (CPU), a graphics processing unit (GPU), a digital signal processor (DSP), a microcontroller, or a specialized accelerator, whether implemented as a single device or distributed across multiple devices.

The term “generative AI model” refers to a machine learning model configured to generate data, such as motion patterns, based on learned relationships from training data, including but not limited to neural network models such as generative adversarial networks (GANs), variational autoencoders (VAEs), transformer-based models, or other models capable of generating new data instances from learned distributions.

The term “prompt” refers to data provided to the generative AI model, including text, numerical parameters, or structured control signals, that specifies or influences the operation of the generative AI model, such as instructing the model which type of data to learn, which reference data to use, or which type of motion pattern to generate.

The term “body capability information” refers to information representing physical characteristics and capabilities of a human body, including but not limited to strength, flexibility, range of motion, body dimensions, joint mobility, and other parameters that affect how the body can perform motion.

The term “motion information” refers to information that represents movement of a subject's body over time, including but not limited to joint positions, joint angles, velocities, accelerations, body posture, timing of motion phases, and any other kinematic or kinetic parameters.

The term “health condition information” refers to information indicating the current or historical physical health status of a subject, including but not limited to medical diagnoses, injury history, pain areas, rehabilitation status, physician-imposed limitations, and measured physiological or orthopedic parameters.

The term “subject” refers to a human individual whose motion, body capability, and health condition information are acquired and for whom an optimal motion pattern is generated and presented.

The term “sensor” refers to a device that detects and outputs information related to physical phenomena associated with the subject, including but not limited to inertial measurement units (IMUs), force sensors, pressure sensors, wearable motion sensors, depth sensors, and other devices capable of measuring body movement or physiological conditions.

The term “camera” refers to an imaging device configured to capture still images or video of the subject, including but not limited to RGB cameras, depth cameras, stereo cameras, and infrared cameras, and to provide image data used to derive the subject's motion information.

The term “learning process” refers to a process in which the processor causes the generative AI model to learn from input data, such as body capability information and motion information, by adjusting internal parameters of the model so that the model captures relationships between inputs and desired outputs.

The term “generation process” refers to a process in which the processor causes the generative AI model to output new data, such as an optimal motion pattern, in response to a prompt and based on the model's previously learned internal parameters.

The term “optimal motion pattern” refers to motion information generated by the generative AI model that is determined to be suitable for a particular subject so that the subject can exhibit a maximum or near-maximum capability while reducing or limiting a risk of injury, taking into account the subject's health condition information and body capability information.

The term “learned data” refers to data that has been used to train the generative AI model, including but not limited to body capability information, motion information, and reference data, and that is implicitly encoded in the internal parameters of the generative AI model after the learning process.

The term “reference data” refers to motion data and related information used as a standard or ideal example in the learning process, including but not limited to motion data of professional or expert athletes that serves as a basis for teaching the generative AI model desirable motion characteristics.

The term “motion data of professional athletes” refers to motion information acquired from individuals recognized as experts or professionals in a sport or physical activity, including high-level performance examples that reflect advanced technique, efficiency, and sport-specific skills.

The term “three-dimensional model” refers to a computer-generated representation of at least part of a human body in three-dimensional space, including a skeleton, mesh, or avatar whose shape and posture can be changed according to motion information such as joint positions and joint angles.

The term “image of a three-dimensional model” refers to visual output, including still images or video, showing the three-dimensional model performing motion over time from at least one viewpoint, and presented on a display device so that a user can visually recognize the motion.

The term “maximum capability” refers to the highest or near-highest level of performance that the subject can achieve under given constraints, considering the subject's body capability information and health condition information, without exceeding medically or physically acceptable limits.

The term “reduced risk of injury” refers to a state in which the likelihood or severity of physical injury associated with a motion pattern is lowered relative to a baseline motion, by adjusting motion parameters such as joint angles, timing, or load in accordance with health condition information and body capability information.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

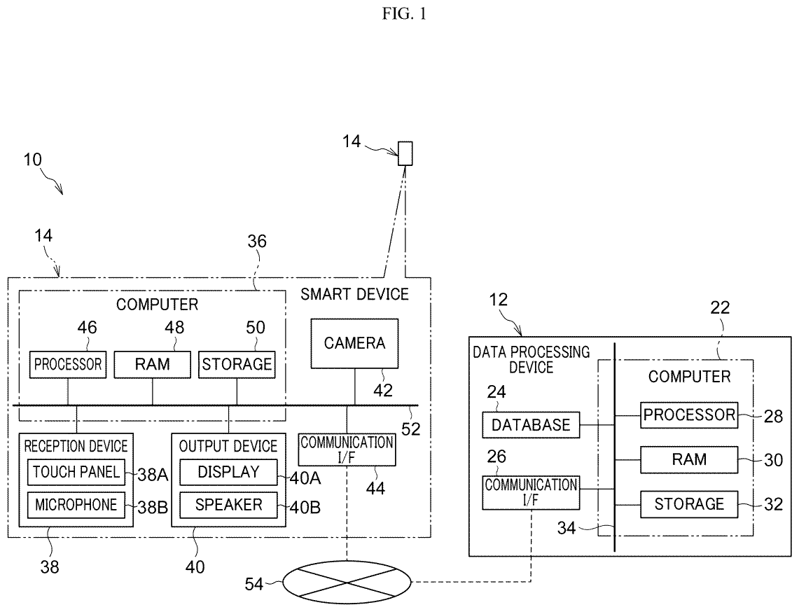

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

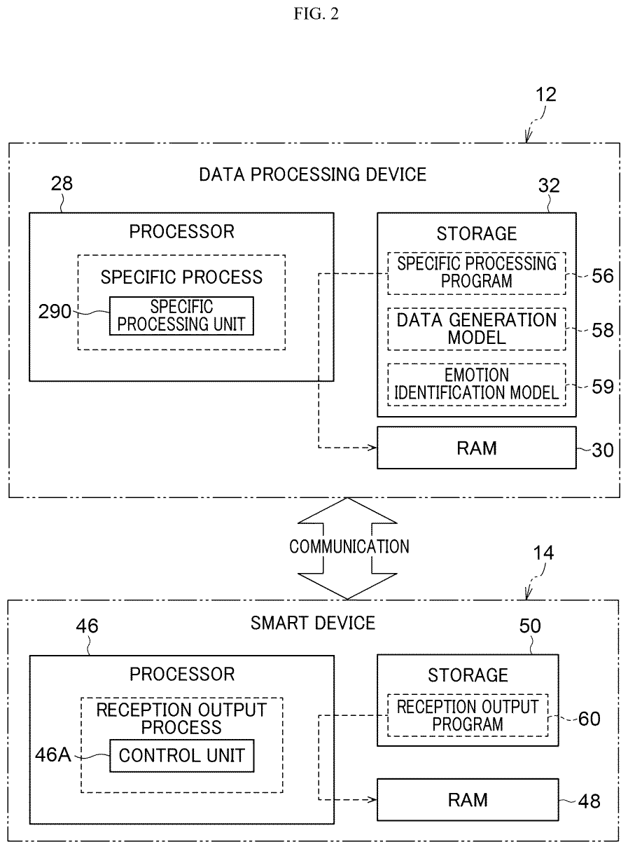

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventional computer-implemented coaching systems for physical training typically apply simple rule-based heuristics or static template matching to motion data captured from users. In many such systems, a computing device acquires motion measurements from sensors, compares the measurements against a fixed set of reference motions, and outputs generic feedback such as “bend the knee more” or “keep the back straight.” These approaches suffer from several technical limitations.

First, conventional systems do not effectively integrate heterogeneous time-series data sources, including high-frequency motion sensor streams, biosignal measurements, and structured health check results. As a result, the internal data representations in memory are often fragmented and poorly normalized, which leads to inefficient use of processor resources and degrades the accuracy of downstream analysis.

Second, conventional systems generally treat motion analysis and motion generation as independent processes. A server may run one algorithm to estimate performance metrics and another, separate algorithm to output suggested motion templates. This disjoint architecture forces repeated data transformations, causes redundant memory access, and increases latency between input capture and feedback generation. Consequently, the user terminal often waits for multiple server-side passes over large time-series data sets, which constrains real-time or near-real-time usability.

Third, existing systems do not exploit generative AI models in a structured, programmatic manner that aligns with the internal data flow of a server. In particular, many systems either (i) use generative models only for natural language recommendations, without generating machine-consumable motion trajectories, or (ii) manually craft user-facing prompts that are not derived from quantified performance metrics or health constraints. This lack of systematic prompt generation tied to internal feature representations prevents the generative models from producing motion patterns that are both biomechanically feasible and personalized to the user's measured capabilities and risks.

Fourth, conventional systems often represent motion feedback as simple scalar scores or textual hints, rather than as a unified three-dimensional motion dataset that can be directly bound to a skeletal animation model. In such systems, the server may output numerical ratings and the client-side application separately constructs animations, leading to duplicated computation, inconsistent visualizations across devices, and unnecessary consumption of communication bandwidth and processing resources.

Therefore, there is a need for an improved computer-implemented system and server architecture that (i) unifies acquisition and preprocessing of multimodal time-series data, (ii) tightly couples an analysis model and a generative AI model via programmatically constructed prompt sentences, and (iii) directly produces optimized three-dimensional motion data and comparative visualization data. Such a system should improve the efficiency of data processing pipelines, reduce end-to-end latency from sensing to visualization, and enhance the quality and machine-readability of the generated optimal motion patterns, thereby improving the overall functioning of the computer system itself in the context of motion analysis and training feedback.

The specific processing by the specific processing unit 290 of the data processing device 12 in Example 1 is realized by the following means.

The present invention provides a server comprising a processor and a memory storing instructions that, when executed by the processor, cause the processor to acquire multimodal time-series data from measurement apparatuses, including at least one wearable measurement device or imaging device, to correct the time-series data to a unified sampling period, to remove noise, to segment the time-series data into a plurality of motion intervals, and to convert each motion interval into a feature sequence including joint angles, angular velocities, load estimation values, and heart rate values; to read, from an external storage, a health check result of a subject and add, as a health status indicator, at least a maximum allowable heart rate, a joint function limitation, and past injury information to the feature sequence; to execute, using an analysis information processing model, an inference process that takes the feature sequence and the health status indicator as inputs, simultaneously estimates a motion capability index and a joint load index for the subject, and outputs a motion pattern that increases capability expression while suppressing injury risk; to programmatically construct, based on an estimation result of the analysis information processing model, a generation instruction prompt sentence in a natural language describing at least subject attributes, a type of physical activity, motion-related problems, and an output format, and to provide the generation instruction prompt sentence together with training data to a generative AI model that is a statistical learning information processing model so as to cause the generative AI model to generate digital data including a sequence of joint angles and associated time information representing an optimal motion pattern; to associate the digital data representing the optimal motion pattern with a three-dimensional skeletal model, to convert the digital data into continuous three-dimensional motion data using inverse kinematics processing and interpolation processing, and to generate video data in which measured motion data of the subject and the optimal motion pattern are displayable for comparison; and to transmit the video data to a terminal device for display. This enables an integrated, computer-implemented pipeline in which the server efficiently transforms heterogeneous sensor and health data into structured feature sequences, tightly couples analysis inference and generative AI processing through dynamically constructed prompt sentences, and directly outputs optimized three-dimensional motion and comparative visualization data, thereby improving computational efficiency, reducing processing latency, and enhancing the technical capability of the computer system to generate precise, machine-readable optimal motion patterns for real-time or near-real-time training feedback.

The term “processor” refers to a hardware computation element, such as a central processing unit, a graphics processing unit, or a specialized logic device, configured to execute instructions stored in a memory to perform data processing operations.

The term “memory” refers to a hardware storage element, such as volatile memory or non-volatile memory, configured to store instructions and data for access by a processor.

The term “information processing model” refers to a computational model implemented in software and/or hardware that processes input data to generate output data according to learned or predefined parameters.

The term “generative AI model” refers to an information processing model based on statistical learning techniques that, in response to input data and a prompt sentence, produces new data instances such as motion trajectories or other structured output that are not merely retrieved from stored templates.

The term “analysis information processing model” refers to an information processing model that receives feature data as input and outputs at least one index or parameter indicative of motion quality, capability, or load, without necessarily generating new motion trajectories.

The term “statistical learning information processing model” refers to an information processing model that has parameters adjusted by training on sample data using machine learning or similar statistical methods, and that applies the trained parameters to infer outputs from inputs.

The term “prompt sentence” refers to a sequence of symbols, including at least natural language text, that encodes instructions, conditions, or context information and is provided to a generative AI model to control or condition its output.

The term “generation instruction prompt sentence” refers to a prompt sentence that specifies at least attributes of a subject, a type of physical activity, motion-related problems, and an output format, and that is used to instruct a generative AI model to generate an optimal motion pattern.

The term “body capability information” refers to data indicating physical characteristics of a subject, including at least strength, flexibility, endurance, balance, or similar biomechanical or physiological attributes.

The term “motion information” refers to data representing physical movement of a subject, including at least kinematic quantities such as positions, velocities, accelerations, orientations, or joint angles over time.

The term “time-series data” refers to data composed of values that are associated with corresponding time points or time intervals and that represent temporal evolution of measured quantities.

The term “measurement apparatus” refers to a hardware arrangement including at least one sensor device configured to acquire physical, physiological, or positional data of a subject, and to output such data to a processor.

The term “wearable measurement device” refers to a measurement apparatus configured to be attached to or worn on a subject's body and to measure at least motion, biosignals, or position during physical activity.

The term “imaging device” refers to an apparatus, such as a camera or depth sensor, configured to capture images or depth data representing a subject's posture or motion.

The term “biosignal” refers to a measurable signal generated by a biological process of a subject, including at least heart rate, muscle activation, respiration, or similar physiological parameters.

The term “posture information” refers to data representing the spatial configuration of body segments or joints of a subject at one or more points in time.

The term “sampling period” refers to a temporal interval between successive data samples in time-series data acquired or processed by a system.

The term “motion interval” refers to a contiguous segment of time-series data corresponding to a single instance or cycle of a motion, such as a single shot, jump, or stride.

The term “feature sequence” refers to an ordered set of feature vectors indexed over time, where each feature vector includes one or more derived quantities such as joint angles, angular velocities, load estimation values, and heart rate values.

The term “joint angle” refers to a quantity representing a relative orientation between two body segments connected at a joint, expressed in one or more rotational degrees of freedom.

The term “angular velocity” refers to a quantity representing a rate of change of orientation of a body segment or joint per unit time.

The term “load estimation value” refers to a computed or inferred quantity that indicates mechanical load, stress, or force applied to a body part or joint of a subject during motion.

The term “health check result” refers to data obtained from medical or health examinations of a subject, including at least clinical measurements, diagnostic findings, or recommended limits.

The term “health status indicator” refers to a set of one or more values derived from a health check result, including at least a maximum allowable heart rate, a joint function limitation, or past injury information, and used as input to an information processing model.

The term “maximum allowable heart rate” refers to a value indicating an upper limit of heart rate that is recommended or prescribed for a subject based on health or safety considerations.

The term “joint function limitation” refers to information indicating a restriction or constraint on movement or load of a joint of a subject, such as a reduced range of motion or reduced strength.

The term “past injury information” refers to data describing previous injuries of a subject, including affected body regions, injury types, or recommended restrictions.

The term “motion capability index” refers to a value or set of values output by an analysis information processing model that quantify a subject's ability to perform a motion, such as power, efficiency, accuracy, or stability.

The term “joint load index” refers to a value or set of values output by an analysis information processing model that quantify mechanical or physiological load applied to one or more joints of a subject during motion.

The term “optimal motion pattern” refers to a motion trajectory or sequence of motions that is determined or generated to improve a subject's capability expression while reducing or maintaining injury risk below a predetermined level.

The term “digital data representing an optimal motion pattern” refers to machine-readable data that encodes at least a sequence of joint angles and associated time information describing an optimal motion pattern.

The term “time information” refers to data specifying at least timing or order of motion states in an optimal motion pattern, such as time stamps, frame indices, or temporal intervals.

The term “subject attributes” refers to data describing characteristics of a subject, including at least age, sex, physical condition, skill level, or dominance of limbs.

The term “type of physical activity” refers to a classification of an activity performed by a subject, such as running, jumping, throwing, kicking, or similar sports-related movements.

The term “motion-related problem” refers to a condition or deficiency in a subject's motion identified by analysis, including at least excessive load, misalignment, instability, or inefficiency.

The term “output format” refers to a specification of the structure or representation of data to be generated by a generative AI model, such as a sequence of joint angles over time or a particular file or data schema.

The term “three-dimensional skeletal model” refers to a digital representation of a body structure comprising interconnected segments and joints in three dimensions, suitable for applying joint rotations and generating animations.

The term “continuous three-dimensional motion data” refers to a set of values over time that define, without temporal gaps, positions and orientations of segments of a three-dimensional skeletal model.

The term “inverse kinematics processing” refers to a computational technique that determines joint parameters required to achieve specified positions or orientations of one or more end effectors or body segments.

The term “interpolation processing” refers to a computational operation that estimates intermediate values between known data points in a time-series or spatial sequence to obtain a smoother or higher-resolution representation.

The term “video data” refers to digital data representing a sequence of images over time, optionally including audio or metadata, suitable for playback by a display device.

The term “measured motion data” refers to motion information obtained directly or indirectly from sensors or imaging devices observing a subject.

The term “terminal device” refers to an endpoint computing device, such as a mobile device, personal computer, or dedicated display unit, configured to receive data from a server and present information to a user.

The term “display device” refers to a hardware component configured to visually present images or video, such as a monitor, screen, or head-mounted display.

The term “reference data set” refers to stored data including motion data of a plurality of performers used as examples or templates in learning or inference processes.

The term “skilled performer” refers to a subject having a comparatively high level of proficiency in a physical activity, whose motion is used as reference data.

The term “teacher data” refers to labeled or target data used during a training process of an information processing model to adjust model parameters.

The term “external storage” refers to any storage apparatus or service, local or network-based, distinct from primary working memory, used to persistently store data such as motion data or health check results.

The term “training data” refers to data used to adjust or refine parameters of a generative AI model or other statistical learning model through a training procedure.

The term “inference process” refers to an execution of an information processing model in which the model receives input data and produces output data using parameters that have been previously determined or trained.

In one embodiment, a system includes a server, at least one terminal, and at least one measurement apparatus worn or used by a user. The user wears a wearable measurement device and optionally uses an imaging device during physical activity, the terminal communicates with the measurement apparatus, and the server executes multiple software modules stored in a memory and executed by a processor. The processor may be implemented by a central processing unit, a graphics processing unit, or a combination thereof. The server runs an operating system such as a general-purpose server operating system and an application stack including a web framework (for example, a typical web application framework), a numerical computation library (for example, a typical array-processing library), and a machine learning framework (for example, a typical tensor-based learning framework).

The measurement apparatus includes, for example, an inertial sensor unit incorporating an accelerometer, a gyroscope, and optionally a magnetometer, a heart-rate sensor, and a position sensor, or an optical imaging camera or depth camera. The terminal includes, for example, a mobile communication device such as a smartphone equipped with a wireless communication module (Bluetooth, wireless LAN, or cellular communication), a display device, and a local storage device. The terminal executes an application program that communicates with the measurement apparatus and the server.

The user wears the wearable measurement device on body segments such as the pelvis, thigh, shank, and foot, or on the upper body, and optionally stands in front of a camera. The measurement apparatus acquires motion-related signals and biosignals in real time during physical activities such as running, jumping, or ball kicking. The terminal receives the raw sensor data via a short-range communication protocol, converts the data into digital time-series samples with associated timestamps, and transmits the time-series data to the server via a packet-based network. The server stores the raw time-series data in a storage device, such as a relational database or a file system, using a structured format such as a row-based or column-based structure with time indices.

The server executes a preprocessing module that normalizes the heterogeneous sensor data into a unified time base. The server resamples the time-series data to a fixed sampling period by performing interpolation of missing samples and decimation of redundant samples. The server applies digital filters implemented, for example, as finite impulse response filters or infinite impulse response filters using a numerical computation library, to remove high-frequency noise while preserving motion-relevant frequency components. The server then segments the unified time-series data into motion intervals by applying event-detection algorithms that detect characteristic changes, such as zero-crossing of joint angular velocity or threshold crossings of acceleration magnitude. The server thus obtains, for each motion interval, a structured array of samples that correspond to one instance of a movement such as a single shot or a single stride.

The server converts each motion interval into a feature sequence. The server calculates joint angles by combining orientation estimates from inertial sensors attached to adjacent body segments and computing relative orientations using rotation matrices or quaternions. The server calculates angular velocities by differentiating joint angles with respect to time. The server estimates load values at joints by applying biomechanical models that approximate joint reaction forces and torques based on limb segment orientations and accelerations. The server associates heart-rate values and other biosignals with the corresponding time indices and adds them as scalar features. The resulting feature sequence is an ordered list of feature vectors, each feature vector including joint angles, angular velocities, load estimation values, and heart rate values at a specific time.

The server reads health check results for the user from an external storage device. The health check results may include, for example, clinical measurements such as resting heart rate, maximum recommended heart rate, findings related to joint stability or cartilage conditions, and records of past injuries such as ligament tears or fractures. The server converts the health check results into a normalized health status indicator by mapping them to scalar or categorical variables, such as a numerical maximum allowable heart rate, a Boolean or categorical joint function limitation indicator, and encoded past injury information.

The server combines the feature sequence and the health status indicator to form an input data structure for an analysis information processing model. In one embodiment, the analysis information processing model is implemented as a neural network architecture specialized for time-series data, such as a model comprising a stack of one or more temporal convolution layers and one or more recurrent layers, such as long short-term memory units or gated recurrent units. The input layer receives, for each time step, the feature vector augmented with health status indicators. The recurrent layers process sequences of feature vectors across time, and one or more dense layers at the output predict, for each motion interval, a motion capability index, such as an efficiency score, power score, or accuracy score, and a joint load index, such as an estimated peak load at a critical joint or an integrated load over the interval. The server trains the analysis information processing model before deployment using training data sets that include feature sequences extracted from historical measurements of multiple subjects, including skilled performers, along with labels that indicate observed performance metrics and joint load estimates derived from high-precision biomechanical analyses. The server uses a loss function that is a weighted sum of errors between predicted and true capability indices and between predicted and true joint load indices. The server uses a gradient-based optimization algorithm, such as stochastic gradient descent with momentum or an adaptive algorithm, to update model parameters. The server may apply data augmentation techniques by adding noise to sensor-derived features, perturbing timing slightly, or mirroring left and right limbs, in order to improve generalization. The server stores trained parameters in a model storage area.

During operation, the server executes an inference process using the trained analysis information processing model. The input feature sequence and health status indicators are passed through the network, and the server obtains, as output, a motion pattern representation that includes, for example, a recommended relative adjustment of joint angles and timing, as well as numerical motion capability indices and joint load indices. In some embodiments, the server also obtains a latent representation vector from an intermediate layer of the analysis model, which encodes key attributes of the user's current motion and physical condition in a compressed form.

The server programmatically constructs a generation instruction prompt sentence for a generative AI model on the basis of the outputs from the analysis information processing model and the health status indicators. The server uses predetermined templates that embed numerical values or categories into natural language. For example, the server generates a prompt sentence such as:

“User: 25-year-old right-footed soccer player with mild instability in the right knee. Data show excessive knee valgus and suboptimal hip rotation during shooting. Goal: maximize shooting accuracy and power while minimizing knee valgus and knee joint load. Task: generate an optimal 3D shooting motion sequence as time-series joint angles for pelvis, hips, knees, and ankles over one shot.”

In another example for a runner, the server generates a prompt sentence such as: “Generate an optimal 3D running gait for a long-distance runner with limited ankle dorsiflexion. The gait should minimize impact forces and improve efficiency. Output a time-series of joint angles for hips, knees, and ankles over one complete stride cycle.” The server then provides the generation instruction prompt sentence, optionally together with a compact representation of the user's current motion pattern, to a generative AI model implemented as a statistical learning information processing model. In one embodiment, the generative AI model is implemented as a sequence-to-sequence neural network comprising an encoder and a decoder, where the encoder processes text tokens of the prompt sentence and encodes them into vector representations, and the decoder outputs, time step by time step, a sequence of joint angle vectors and associated timing values. The generative AI model may include an attention mechanism that allows the decoder to focus on specific parts of the encoded prompt sentence when generating each joint configuration.

The server trains the generative AI model offline with training data that include prompt sentences describing motion context and corresponding target motion trajectories derived from high-quality demonstrations of skilled performers and validated optimal motion sequences. The server tokenizes the prompt sentences and encodes them into embeddings, applies a transformer-based encoder to model language context, and uses a transformer-based or recurrent decoder that emits joint angle vectors. The loss function may include a mean squared error term between generated and target joint angles, a regularization term that penalizes physically impossible joint configurations, and a term that constrains the generated motion to keep joint loads below specified thresholds. The server updates model weights using a gradient-based optimizer.

The server receives, from the generative AI model at runtime, digital data representing an optimal motion pattern as a sequence of joint angle vectors and corresponding time indices. The server checks the generated data for biomechanical plausibility by verifying that joint angles remain within predetermined ranges derived from anatomical constraints and that angular velocities do not exceed safety thresholds. If necessary, the server applies post-processing, such as clipping or smoothing, to enforce constraints.

The server associates the generated joint angle sequence with a three-dimensional skeletal model stored in a 3D asset library. The three-dimensional skeletal model is defined by a hierarchy of bones and joints, with each joint having one or more degrees of rotational freedom. The server uses a 3D graphics tool, such as a general-purpose modeling and animation framework, operated in batch mode via scripting, to apply the generated joint angles to the skeletal rig. The server uses inverse kinematics algorithms to adjust limb segments so that end effectors, such as feet, maintain contact with the ground where required, and uses interpolation algorithms, such as spline interpolation, to generate smooth motion between discrete joint angle samples. The server thereby produces continuous three-dimensional motion data representing the optimal movement.

The server also applies the same or similar processing to the user's measured motion data, mapping measured joint angle sequences to the three-dimensional skeletal model. The server generates video data in which the measured motion and the optimal motion are displayed side by side or overlaid from one or more viewpoints. The server configures virtual cameras in the 3D scene, for example, a frontal view and a lateral view, and renders a sequence of frames showing both motions synchronized in time. The server may additionally generate graphical annotations such as lines indicating joint axes, colored markers highlighting regions of excessive valgus or rotation, and text labels describing corrective cues.

The server transmits the video data to the terminal across the network using a streaming or file-transfer protocol. The terminal receives the video data and displays it on the display device. The user observes differences between their own motion and the optimal motion in the three-dimensional visualization. Because the server has transformed heterogeneous sensor and health data into a unified representation and has generated machine-readable optimal motion trajectories, the user and any client application can rely on precise, quantitative guidance instead of approximate textual feedback.

From a technical standpoint, the use of the described analysis information processing model and generative AI model, tightly integrated via programmatically constructed prompt sentences, improves the functioning of the computer system in several respects. The server minimizes redundant passes over large time-series data by performing preprocessing once, storing intermediate feature sequences, and passing compact feature representations and prompts between models. This reduces memory bandwidth usage and cache misses on the processor. The segmentation and feature extraction scheme reduces input dimensionality while preserving motion-relevant information, which increases inference speed and reduces latency. The specific neural network architectures, loss functions, and constraints produce motion trajectories that align with biomechanical safety criteria while still optimizing for performance, thus improving accuracy and reducing error compared to simple template matching or rule-based systems.

The server also reduces communication load between the server and the terminal by performing computationally intensive analysis and rendering on the server side. Instead of transmitting raw sensor signals or high-dimensional intermediate representations to the terminal, the server transmits compact video data or lightweight meta-information. As a result, the network throughput requirement is reduced, and the terminal can operate with lower processing overhead. The described data structures, including feature sequences, latent vectors, and optimal joint angle sequences, enable efficient caching and reuse for subsequent analysis or retraining.

The described system differs from mere automation of human coaching tasks because the computer system constructs internal, machine-optimized representations and uses trained models with nontrivial architectures to perform transformations that a human would not perform manually, such as high-dimensional feature extraction, joint load estimation from inertial signals, and constrained trajectory generation in joint-angle space. The server applies loss functions and weight updates during training that explicitly encode tradeoffs between capability and safety, which are implemented at a numerical optimization level and cannot be replicated by simple human instructions. The generative AI model, when conditioned on the analysis outputs via prompt sentences, generates new motion patterns that satisfy complex constraints encoded in model parameters, rather than simply suggesting high-level verbal advice.

In another embodiment, the analysis information processing model uses a different architecture, such as a temporal convolutional network without recurrent layers, or a graph neural network that models joints as graph nodes and bones as edges. In yet another embodiment, the generative AI model uses a diffusion-based motion generation architecture, in which the server adds noise to a canonical motion trajectory and trains a denoising network to reconstruct optimal motions from noisy inputs conditioned on encoded prompt sentences. In some variations, the server uses a variational autoencoder to map motion sequences to a latent space, and the prompt sentence influences sampling in the latent space. Each of these alternative architectures still adheres to the same overall data flow: preprocessing and feature extraction from sensor and health data, inference of capability and load indices, prompt construction, generative motion synthesis, skeletal mapping, and comparative visualization. In further embodiments, the terminal may provide local processing capabilities, such as simple on-device filtering or summary visualization, while the server executes the core analysis and generative processes as described. In some cases, the system may be extended to other physical activities or rehabilitation scenarios, where the same data structures and model architectures are reused with different training data and modified loss functions that emphasize rehabilitation objectives. In all such embodiments, the server, the terminal, and the user cooperate to implement a concrete, hardware-tied pipeline in which generative AI models and analysis models, coordinated by prompt sentences, are used to generate and present three-dimensional optimal motion patterns that improve the technical performance of the computer system in motion analysis, processing efficiency, and visualization quality.

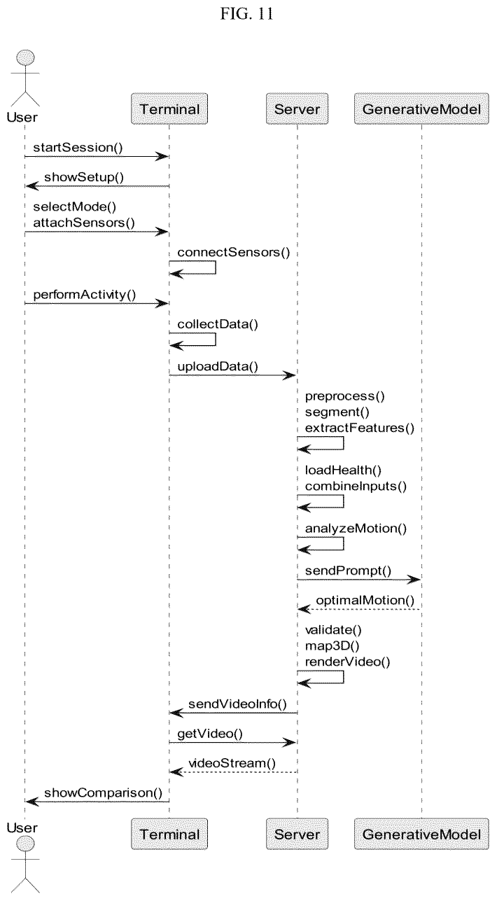

The following describes the processing flow using FIG. 11.

### Step 1:

The user starts a training application on the terminal and prepares the measurement apparatus.

The user wears one or more wearable measurement devices on body segments and optionally positions an imaging device.

Input: No prior digital input is required; the input is the user's selection of a training mode and the physical attachment of sensors.

Output: The terminal holds a training session configuration including sport type, sensor list, and user identifier.

The terminal displays a setup screen, receives user selections (for example, “soccer shooting” mode), and stores the configuration in local memory.

### Step 2:

The terminal establishes a wireless connection with the measurement apparatus.

The terminal uses a wireless communication interface to scan for nearby wearable devices and imaging devices and initiates pairing if needed.

Input: The terminal uses the training session configuration and the list of available wireless devices.

Output: The terminal maintains active wireless connections and registered data channels to the selected measurement devices.

The terminal subscribes to specific sensor data characteristics, such as accelerometer streams and heart-rate notifications, and configures sampling rates.

### Step 3:

The user performs physical activity while sensor data is acquired.

The user executes the target movements, such as repeated shots, jumps, or strides, according to the training mode.

Input: The input is the user's physical motion and physiological state.

Output: The measurement apparatus generates raw analog or digital sensor signals that represent motion and biosignals.

The wearable devices sample inertial, heart-rate, or positional data at predefined intervals and transmit digital data packets to the terminal over the wireless connection.

### Step 4:

The terminal receives and time-stamps raw sensor data.

The terminal listens on wireless channels for incoming packets from each device and associates each packet with a local timestamp.

Input: The input is a stream of device-specific sensor packets from the wearable devices and imaging devices.

Output: The terminal produces a unified raw time-series buffer containing sensor values with associated timestamps and device identifiers.

The terminal decodes binary payloads into numeric values, such as acceleration, angular rate, and heart rate, and appends structured records to an in-memory buffer.

### Step 5:

The terminal structures and temporarily stores the raw time-series data.

The terminal groups the decoded sensor records by device and sensor type and maps device coordinates to standardized body segment labels.

Input: The input is the unified raw time-series buffer with device identifiers and timestamps.

Output: The terminal creates a structured dataset, for example arrays of time, sensor values, and body segment labels, stored in local storage.

The terminal writes the structured data to a local database or file system to handle temporary disconnections and to support retransmission if necessary.

### Step 6:

The terminal prepares and transmits the structured data to the server.

The terminal packages the structured time-series data into messages and applies compression and encryption.

Input: The input is the structured dataset and the training session configuration.

Output: The terminal sends compressed and encrypted data packets to the server over a network protocol and receives a transmission acknowledgment.

The terminal constructs a request that includes session identifiers and user identifiers, compresses the payload, and sends it to a predefined server endpoint using a secure protocol.

### Step 7:

The server receives, authenticates, and stores the raw session data.

The server accepts the incoming request, validates authentication tokens, and verifies integrity checks.

Input: The input is the compressed and encrypted session data and associated headers from the terminal.

Output: The server produces decompressed, validated raw time-series data records stored in a persistent storage system indexed by session ID.

The server decompresses the payload, parses it into internal data structures, checks value ranges and formats, and inserts the records into a database or object store.

### Step 8:

The server performs time-base normalization and noise filtering.

The server loads the raw time-series data for the session and aligns samples from different sensors onto a common time axis.

Input: The input is the stored raw time-series data for all sensors in the session.

Output: The server generates normalized time-series arrays for each body segment and signal type, sampled at a fixed frequency with reduced noise.

The server resamples the data using interpolation where samples are missing and decimation where oversampling occurs, and applies digital filters to remove high-frequency noise that does not contribute to motion analysis.

### Step 9:

The server segments the normalized data into motion intervals.

The server searches the normalized time-series for events indicating the start and end of individual movements.

Input: The input is the normalized multi-sensor time-series arrays.

Output: The server outputs a list of motion intervals, each interval containing a contiguous block of samples corresponding to one movement instance.

The server computes derived signals, such as joint angular velocity magnitude, detects threshold crossings or peaks, and uses these events to define boundaries of motions such as shots, jumps, or strides.

### Step 10:

The server computes kinematic and load-related features for each motion interval.

The server calculates joint angles, angular velocities, and estimated loads using sensor orientations and biomechanical relationships.

Input: The input is each motion interval's normalized time-series data mapped to body segments.

Output: The server generates a feature sequence for each interval, where each time step includes a feature vector that contains joint angles, angular velocities, load estimation values, and biosignal values.

The server converts orientation data to relative joint angles, differentiates joint angles with respect to time for angular velocities, and applies joint load estimation formulas based on limb segment inertia and acceleration.

### Step 11:

The server retrieves and encodes health check information for the user.

The server queries external storage for health records associated with the user and converts them into numeric or categorical indicators.

Input: The input is the user identifier for the session.

Output: The server produces a health status indicator comprising values such as maximum allowable heart rate, joint function limitations, and past injury flags.

The server processes text-based medical notes or structured records into normalized codes and attaches these indicators as additional features to each motion interval.

### Step 12:

The server forms combined input data for an analysis model.

The server merges the feature sequences from motion intervals and the health status indicators into a unified input structure.

Input: The input is the feature sequence for each motion interval and the health status indicator.

Output: The server produces input tensors or equivalent data structures for the analysis information processing model.

The server appends health-related fields to each feature vector, aligns feature dimensions, and arranges the data as sequences to be passed into model layers.

### Step 13:

The server executes inference using the analysis information processing model.

The server loads a trained time-series model and feeds the input sequences through the model.

Input: The input is the prepared input tensors representing feature sequences and health indicators.

Output: The server outputs, for each motion interval, a motion capability index, a joint load index, and internal latent representations of the motion.

The server processes the sequences through layers such as temporal convolutions, recurrent layers, and dense layers, computes intermediate activations, and obtains final predicted indices and latent vectors by applying the model's learned weights.

### Step 14:

The server interprets the analysis results and detects motion deficiencies.

The server examines the motion capability indices and joint load indices and identifies areas where loads exceed thresholds or performance metrics are suboptimal.

Input: The input is the predicted indices and latent representations from the analysis model.

Output: The server generates structured diagnostic information indicating specific joints, phases of motion, and types of deficiencies.

The server compares predicted joint load indices against health-based limits, flags excessive valgus angles or high-impact landings, and records these findings as structured problem descriptions.

### Step 15:

The server constructs a generation instruction prompt sentence for the generative AI model.

The server translates the diagnostic information, motion context, and user attributes into a natural-language description following predefined templates.

Input: The input is the diagnostic information, motion capability indices, joint load indices, health indicators, and user attributes.

Output: The server produces a prompt sentence that encodes conditions, goals, and required output format for the generative AI model.

The server composes text such as:

“User: 25-year-old right-footed soccer player with mild instability in the right knee. Data show excessive knee valgus and suboptimal hip rotation during shooting. Goal: maximize shooting accuracy and power while minimizing knee valgus and knee joint load. Task: generate an optimal 3D shooting motion sequence as time-series joint angles for pelvis, hips, knees, and ankles over one shot.”

### Step 16:

The server sends the prompt sentence to the generative AI model and requests an optimal motion pattern.

The server invokes a generative AI model interface with the constructed prompt sentence and optional latent representations.

Input: The input is the generation instruction prompt sentence and, optionally, a compact latent code encoding the user's current motion.

Output: The server receives digital data representing an optimal motion pattern as a time-ordered sequence of joint angle vectors and timing values.

The server passes the text prompt through a tokenizer, sends encoded text to the model, and waits for the model to emit a sequence of joint configurations that satisfy the described constraints.

### Step 17:

The server validates and post-processes the generated optimal motion sequence.

The server checks that each generated joint angle lies within anatomically plausible ranges and that abrupt changes do not exceed safe angular velocity limits.

Input: The input is the generated sequence of joint angles and timing values from the generative AI model.

Output: The server outputs a corrected joint angle sequence and timing sequence suitable for animation.

The server applies constraint-checking routines, clips or smooths any out-of-range values, and, if necessary, interpolates between keyframes to obtain a smooth trajectory.

### Step 18:

The server maps the optimal motion sequence onto a three-dimensional skeletal model.

The server associates each joint angle in the sequence with corresponding joints in a 3D skeletal rig stored in a graphics asset.

Input: The input is the corrected joint angle sequence, timing sequence, and the skeletal model definition.

Output: The server produces continuous three-dimensional motion data that specify positions and orientations of skeletal segments over time.

The server computes joint transformations in 3D space, applies inverse kinematics to adjust end-effector positions, and interpolates motions between discrete samples to create a frame-by-frame animation.

### Step 19:

The server generates comparative video data showing measured motion and optimal motion.

The server uses a rendering environment to animate both the measured motion and the generated optimal motion within a single scene.

Input: The input is the 3D motion data for the user's measured motion and the 3D motion data for the optimal motion.

Output: The server outputs video data or an animation file where both motions are visible from one or more viewpoints.

The server positions virtual cameras, renders frames from each camera, overlays annotations that highlight differences, and encodes the rendered frames into a video stream.

### Step 20:

The server transmits the video data to the terminal.

The server prepares a response message including a link or payload containing the rendered comparison video.

Input: The input is the video data and the session identifier.

Output: The server returns a response to the terminal that enables download or streaming of the comparison video.

The server stores the video in a content storage system if necessary, generates an access token or URL, and includes this information in the response.

### Step 21:

The terminal retrieves and presents the comparison video to the user.

The terminal requests the video according to the server response, downloads or streams it, and plays it on the display.

Input: The input is the server response containing video access information.

Output: The terminal displays synchronized images of the measured motion and the optimal motion to the user.

The terminal provides controls such as play, pause, and scrubbing, and may show side-by-side or overlaid views so that the user can visually inspect and compare the motion patterns.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

Conventional motion-guidance systems for physical work and training environments generally rely on static rules, pre-authored motion templates, or simple threshold-based evaluations of sensor signals. These systems typically process sensor data locally and compare the data to fixed reference ranges, for example, by checking whether a joint angle exceeds a predetermined limit. Such approaches suffer from several technical limitations. First, conventional systems are not capable of dynamically generating subject-specific optimal motion patterns in real time based on heterogeneous data sources including body capability information, health state information, task context, and time-series motion information. The underlying processing pipelines are not designed to encode these different data types into a unified representation that a machine-learning model can consume and update online. As a result, the generated feedback remains coarse and generic, and cannot effectively adapt to the changing physical state of the user.

Second, existing systems generally treat three-dimensional visualization as a separate, manually configured stage. A human operator often needs to map processed sensor data to a three-dimensional avatar or template using pre-defined scripts or offline tools. This separation between analysis and visualization leads to latency, inconsistency, and additional configuration overhead, and prevents the system from providing a tightly coupled, low-latency feedback loop between sensor acquisition, computation of optimal form, and three-dimensional display on a portable display apparatus.

Third, although generative AI models have become capable of producing sophisticated text and data outputs, conventional motion-guidance systems do not exploit such models as part of the core computational pipeline. In typical usages, a generative AI model, if used at all, is queried with manually written prompts that are not derived from structured sensor data or model outputs, and the generative AI model is not integrated into the main feedback loop. This results in inefficient use of computational resources and prevents the system from automatically generating tailored motion guidance texts that are synchronized with optimal motion patterns computed from raw sensor data.

Fourth, many existing architectures are not optimized for iterative feedback in which updated sensor data, updated optimal motion patterns, three-dimensional visualization, and generated textual guidance are continuously cycled. The lack of such an integrated feedback loop leads to redundant data transfers, repeated ad-hoc conversions between data formats, and increased processing latency. This degrades both the responsiveness and accuracy of the system as a whole.

Accordingly, there is a need for an improved computer-implemented system and method that (i) unifies acquisition and preprocessing of biological and motion information, (ii) programmatically constructs prompt sentences encoding such information to drive learning and generation processes in a generative AI model, (iii) converts the generated optimal motion patterns directly into numerical representations suitable for three-dimensional skeletal animation, and (iv) streams both the three-dimensional motion image data and synchronized motion guidance text to a portable display apparatus, all within a continuous feedback loop. Such an integrated architecture would improve the functioning of the computer system itself by reducing latency, eliminating manual configuration steps, and enabling context-aware, user-specific guidance to be computed and displayed in real time.

The specific processing by the specific processing unit 290 of the data processing device 12 in Application Example 1 is realized by the following means.

The present invention provides a server comprising a processor configured to acquire biological information, health state information, and time-series motion information of a user from at least one information acquisition apparatus including a sensor or an imaging apparatus; to generate body capability information and motion information from the acquired biological information and motion information; to automatically construct a first prompt sentence that instructs a generative AI model to perform a learning process using the body capability information and the motion information and to cause the generative AI model to execute the learning process based on the first prompt sentence; to acquire, during execution of a task, updated health state information of the user and motion information of the user; to automatically construct a second prompt sentence that instructs the generative AI model to generate an optimal motion pattern in which the user exerts a maximum capability in the task while reducing a risk of fatigue and injury, the second prompt sentence being based on at least the updated health state information and the updated motion information, and to cause the generative AI model to execute a generation process of the optimal motion pattern based on the second prompt sentence; to convert the optimal motion pattern output from the generative AI model into numerical data including a sequence of joint angles and a sequence of body-part trajectories; to generate three-dimensional motion image data by applying the numerical data to a three-dimensional display object having a skeletal structure and a surface shape in a virtual space; to transmit the three-dimensional motion image data to a portable display apparatus via a communication line so that the portable display apparatus displays the three-dimensional motion image data in a manner superimposed on a real space for real-time comparison by the user; to acquire, as feedback, further updated motion information of the user who has corrected an own motion based on the display and to repeatedly execute generation of the optimal motion pattern and generation of the three-dimensional motion image data; and to automatically construct a third prompt sentence that instructs the generative AI model to generate a motion guidance text for the user based on at least one of the body capability information, the health state information, task content information, and the optimal motion pattern, to obtain the motion guidance text generated by the generative AI model, and to transmit the motion guidance text to the portable display apparatus. This enables an integrated, computer-implemented feedback loop in which heterogeneous sensor and health data are programmatically encoded into prompt sentences for the generative AI model, optimal motion patterns are generated and transformed into three-dimensional motion image data, and synchronized textual guidance is delivered to the portable display apparatus in real time, thereby improving the technical performance, responsiveness, and adaptability of the motion-guidance system.

The term “biological information” refers to data representing physical characteristics or physiological parameters of a human subject, including at least one of body size, body composition, strength, flexibility, endurance, heart rate, and similar measurable attributes.

The term “health state information” refers to data indicating a current or historical health condition of a user, including at least one of medical examination results, injury records, physical limitations, fatigue levels, and other medically relevant status indicators.

The term “motion information” refers to time-series data representing movement of at least part of a user's body, including at least one of joint angles, positions, velocities, accelerations, and trajectories of body segments.

The term “information acquisition apparatus” refers to an electronic apparatus configured to detect and output biological information or motion information, including at least one of a sensor device, an imaging device, a wearable device, or a combination thereof.

The term “sensor” refers to an electronic device that measures a physical quantity related to a user's body or motion, including at least one of an inertial sensor, a force sensor, a position sensor, a pressure sensor, or a physiological sensor.

The term “imaging apparatus” refers to a device that acquires image data or video data of a user or an environment, including at least one of a camera, a depth sensor, or an optical motion-capture device.

The term “body capability information” refers to data derived from biological information and optionally health state information, indicating a capability profile of a user, including at least one of maximum safe load, range of motion, muscular strength, flexibility, and endurance.

The term “task content information” refers to data describing a task to be performed by a user, including at least one of task type, required movements, required load handling, workspace configuration, and target positions of manipulated objects.

The term “generative AI model” refers to a machine-implemented information processing model configured to generate outputs such as text or numerical data by performing inference based on input data, including at least one of a neural network-based language model and a neural network-based sequence generation model.

The term “prompt sentence” refers to machine-readable text data supplied to a generative AI model, the text data being constructed to instruct the generative AI model to perform at least one of a learning process and a generation process using specified input information.

The term “learning process” refers to a computation in which model parameters of a generative AI model are updated or adapted based on training data or fine-tuning data, in order to improve performance of subsequent generation or inference.

The term “generation process” refers to a computation in which a generative AI model produces an output such as an optimal motion pattern or motion guidance text based on given input data and a prompt sentence, without necessarily updating model parameters.

The term “optimal motion pattern” refers to data representing a sequence of body postures and movements that satisfies one or more criteria, including that a user exerts a high level of task performance while a risk of fatigue or injury is reduced.

The term “numerical data including a sequence of joint angles and a sequence of body-part trajectories” refers to a structured representation of an optimal motion pattern, in which joint angles and positions of body parts are specified as time-series values in at least one coordinate system.

The term “three-dimensional display object” refers to a digital representation of an object in a virtual three-dimensional space, the object having at least a skeletal structure for defining joint relationships and a surface shape for visual rendering.

The term “skeletal structure” refers to a hierarchical arrangement of virtual bones or joints defining relative spatial relationships used to control deformation or motion of a three-dimensional display object.

The term “surface shape” refers to a mesh, polygon set, or other geometric representation defining an outer appearance of a three-dimensional display object for rendering on a display device.

The term “three-dimensional motion image data” refers to data defining time-varying states of a three-dimensional display object, including at least one of key frames, animation curves, and per-frame pose information, enabling rendering of a moving three-dimensional image.

The term “portable display apparatus” refers to a user-worn or hand-held electronic apparatus configured to display images, including at least one of a head-mounted display, smart glasses, a portable terminal, or a tablet device.

The term “communication line” refers to a wired or wireless communication path by which data is transmitted between the server and another apparatus, including at least one of a local network, a wide-area network, a wireless network, and the Internet.

The term “real space” refers to a physical environment surrounding a user, as opposed to a purely virtual space rendered by a computer.

The term “superimposed on a real space” refers to a display mode in which three-dimensional motion image data is overlaid on an image of a real environment or directly in the user's field of view such that virtual content appears to coexist with physical objects.

The term “feedback process” refers to a repeated computational cycle in which updated motion information of a user is acquired, an optimal motion pattern is recalculated, and corresponding three-dimensional motion image data and guidance information are regenerated.

The term “motion guidance text” refers to a natural-language textual output generated by a generative AI model, the output including at least one of instructions, recommendations, or cautions relating to a user's motion or posture.

The term “time-series data” refers to data in which measurements or values are associated with respective time points or time intervals, enabling analysis of temporal changes in biological or motion information.

In one embodiment, a server cooperates with at least one terminal and at least one user-worn information acquisition apparatus to implement the claimed system. The server includes at least one general-purpose processor, a memory, a non-volatile storage device, a network interface, and optionally a graphics processing unit. The server executes an operating system such as a server-class operating system and application software including a database management system, a numerical computation framework such as a tensor computation framework, a machine-learning framework such as a deep-learning library, and a three-dimensional graphics engine such as a game engine or a server-side rendering engine. The terminal includes a portable display apparatus such as a head-mounted display, smart glasses, or a handheld terminal. The terminal includes an integrated processor, a graphics processing unit or graphics circuitry, a wireless communication unit such as a wireless local area network module or a cellular communication module, and a display panel. The terminal executes a runtime environment such as an application platform, a web runtime, or a dedicated client application capable of decoding and rendering three-dimensional motion image data.

The user wears or carries an information acquisition apparatus that includes at least one inertial sensor, force sensor, optical sensor, or imaging apparatus. For example, the user may wear an inertial measurement unit on multiple body segments, a motion-capture suit, or a body-mounted camera. The information acquisition apparatus is configured to generate time-series sensor data representing joint angles, accelerations, angular velocities, and positions of body segments. The information acquisition apparatus transmits the sensor data to the server over a wired or wireless communication link.

The server stores, in the storage device, a plurality of data structures including: (i) a user profile table that holds body capability information and health state information of each user; (ii) a task definition table that holds task content information specifying types of physical tasks, required load handling, typical motion patterns, and workspace geometry; (iii) a motion log data store that holds raw sensor measurements and preprocessed motion information as time-series records; (iv) model configuration data including network architecture definitions, normalization parameters, and feature-extraction definitions; and (v) a prompt template repository storing prompt sentence templates for queries to a generative AI model.

The server generates a set of software modules implementing a data ingestion component, a feature-extraction component, a generative AI interface component, a motion-synthesis component, a three-dimensional animation component, and a guidance-generation component. Each component runs as one or more processes or services on the server. The data ingestion component configures network sockets, message brokers, or application-layer protocols such as HTTP or WebSocket to receive time-stamped sensor packets from the information acquisition apparatus. The server converts, for example, a binary sensor packet into a normalized internal format such as a vector of floating-point values representing each joint angle, acceleration, and angular velocity. The server writes each converted sample into the motion log data store along with metadata fields including a user identifier, sensor identifier, and task identifier.

The server uses the feature-extraction component to transform the raw motion information into a representation suitable for a generative AI model. In one embodiment, the server computes joint angle velocities by finite-difference operations over consecutive samples, computes joint angle accelerations by a second-order finite difference, and estimates joint torques using a kinematic model of the human body stored as a set of link lengths and mass parameters. The server aggregates a fixed-length window of samples, such as 2 seconds or 3 seconds, into a three-dimensional tensor with dimensions [time steps]×[joints]×[features per joint]. The server normalizes each feature dimension using precomputed means and standard deviations stored in the model configuration data, thereby reducing the dynamic range and improving the numerical stability of the subsequent neural-network computations.

The server implements the generative AI interface component as a module that constructs and sends prompt sentences to a generative AI model exposed as a service. In one embodiment, the generative AI model is a transformer-based language and sequence-generation model deployed on a separate computation node or provided as a remotely hosted service. The server does not simply send unstructured text; instead, the server programmatically composes the prompt sentence based on structured data taken from the user profile table, the task definition table, and the feature-extraction component.

For example, the server generates a learning-related prompt sentence such as: “The system has collected joint angle and velocity data from multiple workers performing repetitive lifting tasks. Each record contains normalized joint angles for the spine, hips, knees, and shoulders sampled at 100 Hz, along with corresponding labels indicating whether the motion pattern is efficient and safe. Use these signals as training data to refine your internal representation of safe and efficient lifting motions for different body capability profiles.”

The server transmits such a prompt sentence together with a compact representation of the features, for example as a tokenized description or embedded metadata, to the generative AI model. The generative AI model updates its internal representations or generates internal task-specific embeddings based on these instructions. By encoding the sensor-level statistics, body capability information, and task description into a structured prompt sentence, the server enables the generative AI model to adapt to the domain of ergonomic motion control. The server also constructs a generation-related prompt sentence for producing an optimal motion pattern. For example, the server generates a prompt sentence such as:

“The worker is 175 cm tall and has limited lower-back flexibility. Current sensor data shows lumbar flexion angles reaching 55 degrees when lifting a 15 kg object from the floor to a waist-height surface. Generate a time-series description of joint angles for the spine, hips, knees, and ankles for an alternative motion pattern that reduces peak lumbar flexion by at least 15 degrees while maintaining the ability to complete the lift.”

The server embeds, into this prompt sentence, numerical parameters such as the current joint-angle statistics, the target constraints, and the user's body capability limits. The server sends this prompt sentence to the generative AI model and receives, as output, a structured sequence description that can be parsed into an optimal motion pattern. In one embodiment, the generative AI model outputs a sequence of tokens that describe joint angles in discrete time steps, which the server then decodes into a numerical sequence of joint configurations. In order to reduce computational overhead and network latency, the server uses a specific encoding scheme for the motion pattern. For example, the server quantizes joint angles into a fixed precision and encodes them as integer indices referencing a table of basis poses. The server stores this basis-pose table in memory and reconstructs full joint angles on the server side by table lookup and interpolation. This approach reduces the size of data transmitted between the generative AI model and the server, thereby decreasing communication load and enabling faster interaction cycles.

The server uses the motion-synthesis component to combine the generated optimal motion pattern with a kinematic model of the user's body. The server computes forward kinematics by applying the sequence of joint angles to a skeletal structure representing the human body as a hierarchy of bones. Optionally, the server applies temporal interpolation and smoothing algorithms, such as cubic spline interpolation or low-pass filtering of joint trajectories, to reduce abrupt changes and to ensure that the synthesized motion is physically plausible. These algorithms are implemented as numerical operations on arrays of joint angles and positions, and they improve numerical stability and smoothness of the final animation.

The server uses the three-dimensional animation component to bind the processed joint trajectories to a three-dimensional display object. In one embodiment, the server maintains a rigged three-dimensional avatar with a predefined skeletal structure and a polygonal surface mesh. The server maps each joint angle in the optimal motion pattern to a corresponding joint in the skeletal structure and computes the pose matrix for each frame. The server stores the sequence of pose matrices in an animation buffer. The server then encodes the three-dimensional motion image data into a format suitable for streaming to the terminal, such as a stream of pose matrices or a compressed animation clip.

By generating three-dimensional motion image data on the server, the system reduces the processing burden on the terminal. The terminal only needs to decode the motion image data and apply it to a local copy of the avatar model. This division of labor improves frame rate and responsiveness on resource-constrained terminals, and also reduces the need to transfer large mesh or texture assets during each session.

The server also generates a motion guidance text using the generative AI model. For example, in response to an internal state that indicates excessive lumbar flexion and insufficient knee flexion, the server constructs a prompt sentence such as:

“The worker is performing a repetitive task lifting 10 kg boxes from the floor to a chest-height conveyor. Sensor analysis shows that the knees are flexed only 10 degrees while the lower back is flexed 50 degrees at peak load. Provide step-by-step instructions to shift more motion to the hips and knees and reduce lower-back flexion, using clear and concise language suitable for a worker.”

The server sends this prompt sentence and the associated motion-analysis parameters to the generative AI model. The generative AI model returns a natural-language motion guidance text, which the server stores and forwards to the terminal. The server optionally segments the guidance text into shorter units, for example, bullet points that align with phases of the synthesized motion pattern (start posture, lifting phase, placement phase), enabling the terminal to synchronize textual tips with corresponding frames of the three-dimensional animation.

The terminal receives the three-dimensional motion image data and the motion guidance text from the server via the wireless communication unit. The terminal decodes the pose information and uses its graphics processing unit to render the avatar in three dimensions. In an augmented-reality configuration, the terminal superimposes the avatar in the user's field of view so that the avatar appears at a position relative to the user's body or the workspace. The terminal renders the avatar's motion in temporal alignment with the actual task being performed by the user. For example, when the user begins a lifting motion, the terminal starts playback of the corresponding portion of the optimal motion pattern.

The terminal also presents the motion guidance text as a visual overlay or as audio via a text-to-speech engine. The user can read or listen to the guidance while observing the avatar's movement. By synchronizing the textual tips with the three-dimensional motion image, the system provides multi-modal guidance that improves user comprehension and compliance with the suggested motion corrections.

The user views the avatar and guidance while performing the physical task. The user modifies body posture and movement in response to the displayed optimal motion pattern and motion guidance text. The information acquisition apparatus continues to capture updated motion information and transmits this to the server. The server thus maintains a continuous feedback loop, repeatedly updating the motion analysis, prompting the generative AI model for refined optimal patterns or additional guidance, and updating the three-dimensional animation and text output delivered to the terminal.

From a technical perspective, the system improves computer functionality in several ways. By encoding heterogeneous data (sensor signals, health state information, task content information) into structured prompt sentences, the server enables the generative AI model to operate on a richer and more context-aware representation than simple rule-based systems. The server's feature-extraction and normalization pipeline reduces noise and variability in sensor data, allowing more accurate and stable inference of optimal motion patterns. The separation between heavy computation on the server and lightweight rendering on the terminal reduces processing load on the portable device and improves battery life and thermal performance.

Furthermore, the server implements a specific neural-network architecture and training method that differs from conventional systems. In one embodiment, the generative AI model for optimal motion pattern generation includes: (i) an encoder that maps tokenized descriptions of body capability and task constraints into a latent vector; (ii) a temporal module such as a sequence model that predicts joint angles over discrete time steps; and (iii) a decoder that outputs structured descriptions of joint configurations. The server trains this model using a loss function that combines a reconstruction error term (difference between predicted and expert joint angles) and a penalty term for unsafe postures (for example, exceeding a maximum flexion angle). The server applies gradient-based optimization, such as stochastic gradient descent or adaptive methods, and performs data augmentation such as temporal scaling or mirroring of motion sequences. These training details ensure that the model learns to generate motions that satisfy ergonomic constraints while preserving task effectiveness.

In contrast to a simple automation of human analysis, the server uses non-intuitive rules and multi-objective optimization within the neural network. For example, the model learns to trade off between joint torque minimization and task completion time, or to shift load between different joints based on body capability information. Such trade-offs are encoded in the loss function and in the architecture's attention mechanisms, and they cannot be trivially replicated by manual rule sets. As a result, the system can discover movement strategies that reduce injury risk while maintaining productivity, which conventional systems cannot efficiently compute.

The specific data flow and modular structure also contribute to technical effects. Because the server keeps a compact intermediate representation of motion features and optimal patterns in memory, the system can recompute updated guidance with low latency when new sensor data arrives. The server can selectively update only parts of the optimal motion pattern that differ significantly from the previous cycle, thereby reducing redundant computation and network traffic. This selective update mechanism lowers communication load between the server and the terminal and allows the system to maintain a high frame rate and responsiveness. Alternative embodiments are possible within the same inventive concept. For example, the server can use a different type of generative AI model, such as a variational autoencoder or a diffusion-based sequence generator, instead of a transformer. The feature-extraction component can be adapted to other sensor modalities, such as pressure-sensitive insoles for gait analysis or electromyography sensors for muscle activation patterns. The three-dimensional animation component can use different avatar types, ranging from highly realistic human models to simplified stick figures, depending on the capability of the terminal. The motion guidance text can be generated in multiple languages by adjusting the prompt sentences and the output format.

In another embodiment, the server can integrate a local motion-comparison module on the terminal. In this case, the server still computes and transmits the optimal motion pattern, but the terminal computes the difference between the user's current pose and the target pose frame-by-frame. The terminal then overlays graphical indicators, such as colored arrows or deviation bars, directly in the user's field of view. This variation offloads some numerical computation to the terminal while still relying on the server for generative AI processing and three-dimensional motion synthesis.

Through these configurations, the system provides a concrete, hardware-linked implementation that goes beyond abstract data processing. The server, the terminal, and the user-worn information acquisition apparatus cooperate to measure actual human motion, compute optimized motion patterns using specific neural-network architectures and algorithms, and control the rendering hardware of the terminal to display synchronized three-dimensional animations and textual guidance. This integrated arrangement improves the technical performance of the computer-implemented motion-guidance system in terms of processing speed, motion-prediction accuracy, communication efficiency, and overall responsiveness.

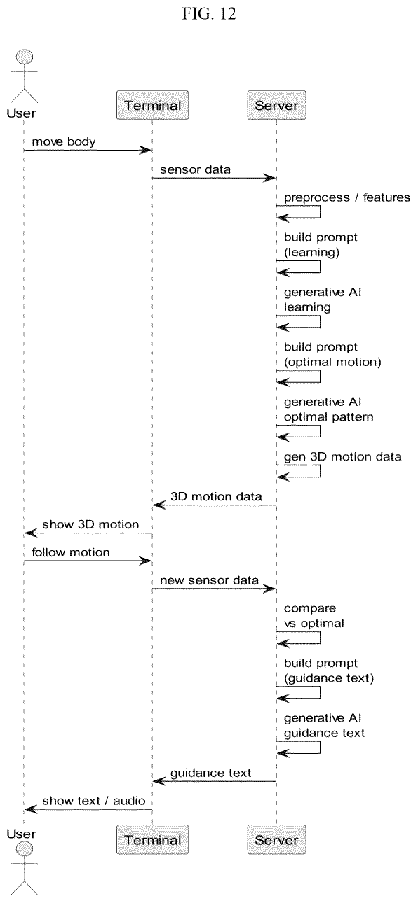

The following describes the processing flow using FIG. 12.

### Step 1:

The server acquires raw sensor data from the information acquisition apparatus.

The server receives, as input, time-stamped packets including joint angles, accelerations, angular velocities, and optionally image frames from at least one sensor or imaging apparatus worn or used by the user.

The server parses each packet, verifies a packet header, decodes numerical values into floating-point arrays, and discards corrupted or incomplete records.

The server outputs normalized raw sensor samples, each tagged with a user identifier, sensor identifier, and task identifier, and stores them in a motion log or buffer for subsequent processing.

### Step 2:

The server generates motion information from the raw sensor data.

The server takes, as input, a sequence of normalized raw sensor samples for a recent time window, for example the last 2-3 seconds of data for a given user and task.

The server computes joint angle velocities using first-order finite differences between consecutive samples and computes joint angle accelerations using second-order finite differences.

The server optionally estimates joint torques by applying a kinematic and dynamic model of the body, combining joint angles, segment lengths, and mass distribution data obtained from the user's body capability information.

The server outputs motion information as time-series feature vectors for each time step, including, for example, joint angles, velocities, accelerations, and estimated torques.

### Step 3:

The server generates body capability information and consolidates user-specific context.

The server uses, as input, biological information (such as height, weight, strength scores), health state information (such as injury history, flexibility limits), and stored user profile records.

The server computes derived capability parameters, such as maximum recommended load per joint, maximum safe flexion angles, and endurance scores, by applying simple arithmetic, lookup tables, and rule-based constraints.

The server outputs a structured body capability profile for the user, represented as a parameter vector or record used by later processing steps.

### Step 4:

The server constructs a feature tensor for AI processing.

The server uses, as input, the motion information (time-series features) and the body capability profile.

The server normalizes each feature dimension using precomputed mean and standard deviation values, performs clipping of extreme outliers, and concatenates capability parameters to each time step or to a separate conditioning vector.

The server reshapes the resulting data into a feature tensor of a fixed shape, for example [T×J×F] for time steps, joints, and features per joint, and possibly [C] for capability features.

The server outputs this feature tensor as the standardized representation supplied to the generative AI model.

### Step 5:

The server composes a learning-related prompt sentence for the generative AI model.

The server uses, as input, statistics of the feature tensor (such as average joint angles, variance, label distributions), meta-information about the task, and stored examples of expert motion patterns.

The server fills a stored template with these values to construct a prompt sentence, for example:

“The system has collected normalized joint angle and velocity sequences for the spine, hips, knees, and shoulders from expert workers and novice workers performing lifting tasks. Use these signals to refine your internal representation of safe and efficient lifting motions for users with varying body capabilities.”

The server outputs the composed prompt sentence and any accompanying compact descriptors that identify where the underlying training data are stored or how they are structured.

### Step 6:

The server causes the generative AI model to perform a learning process.

The server uses, as input, the learning-related prompt sentence and a subset of feature tensors corresponding to labeled expert and non-expert motions.

The server encodes the prompt sentence into tokens, sends the tokens and a description of the feature tensors to the generative AI model via a network API, and triggers a learning or fine-tuning operation on the model side.

The server receives, as output, a confirmation or updated model reference indicating that the internal parameters or task-specific embeddings of the generative AI model have been adapted to the motion-guidance domain.

### Step 7:

The server composes a generation-related prompt sentence for an optimal motion pattern.

The server takes, as input, the feature tensor representing the current user's recent motion, the body capability profile, and the current task description (for example, lifting, carrying, overhead assembly).

The server analyzes the feature tensor to identify deviations from recommended ranges and calculates constraints, such as “reduce lumbar flexion by at least X degrees” or “limit shoulder abduction under a threshold.”

The server composes a prompt sentence, for example:

“The worker is performing repetitive lifting of a 15 kg object from floor level to a waist-height platform. Current motion shows lumbar flexion peaks at 55 degrees and knee flexion peaks at 20 degrees. Generate a time-series of joint angles for spine, hips, knees, and ankles that reduces lumbar flexion peaks to 35-40 degrees and increases knee flexion peaks to at least 45 degrees while still completing the lift.”

The server outputs this generation-related prompt sentence and a compact description of the current motion statistics and constraints.

### Step 8:

The server requests an optimal motion pattern from the generative AI model.

The server uses, as input, the generation-related prompt sentence and identifiers referencing the current user, task, and constraint set.

The server tokenizes the prompt sentence, sends the tokens to the generative AI model, and specifies that the output should be a structured description of a sequence of joint angles over discrete time steps.

The generative AI model returns, as output, a sequence specification such as discrete joint-angle values or parameterized motion segments, which the server decodes into a numerical optimal motion pattern for all relevant joints and time steps.

### Step 9:

The server converts the optimal motion pattern into numerical data for animation.

The server uses, as input, the optimal motion pattern obtained from the generative AI model.

The server maps each token or symbolic description to actual numerical joint angles, applies unit conversions if needed, and interpolates between coarse key poses to reach the desired temporal resolution.

The server organizes the results into arrays representing a sequence of joint angles and a sequence of body-part trajectories relative to a defined coordinate system.

The server outputs numerical data suitable for direct application to a three-dimensional skeletal structure.

### Step 10:

The server performs smoothing and interpolation on the motion data.

The server takes, as input, the raw numerical joint sequences generated in Step 9.

The server applies temporal interpolation (for example, linear interpolation or spline interpolation) between key frames and applies smoothing filters (for example, low-pass filtering) to reduce jerk and discontinuities.

The server ensures that joint angles remain within biomechanical limits obtained from the body capability profile by clamping or adjusting out-of-range values.

The server outputs a refined sequence of joint angles and trajectories that is smooth, continuous, and biomechanically plausible.

### Step 11:

The server generates three-dimensional motion image data.

The server uses, as input, the refined joint-angle sequences and a stored three-dimensional avatar model with a skeletal structure and surface mesh.

The server computes, for each time step, transformation matrices for each joint in the skeleton using forward kinematics, and applies skinning algorithms to deform the mesh accordingly.

The server encodes the resulting per-frame poses and, optionally, vertex positions into an animation format or a stream of pose matrices, along with timing information such as frame rate and duration.

The server outputs three-dimensional motion image data that represent the optimal motion pattern as a playable animation sequence.

### Step 12:

The server transmits the three-dimensional motion image data to the terminal.

The server takes, as input, the encoded three-dimensional motion image data and session identifiers for the target user and terminal.

The server segments the data into packets, adds headers including sequence numbers and timestamps, and transmits the packets through a network interface using a communication protocol such as TCP or UDP over a wireless or wired network.

The server may compress the data or apply streaming techniques to minimize latency and bandwidth usage.

The server outputs a continuous data stream or downloadable animation object that the terminal can decode and render.

### Step 13:

The terminal receives and decodes the three-dimensional motion image data.

The terminal uses, as input, the data stream or animation object sent by the server.

The terminal reassembles packets into a complete animation sequence, validates integrity using checksums or sequence numbers, and decodes pose matrices or key-frame data into an internal animation buffer.

The terminal allocates memory for storing the animation and associates the animation with a local avatar model matching the skeletal structure used by the server.

The terminal outputs a ready-to-play three-dimensional animation object within its rendering engine.

### Step 14:

The terminal renders the optimal motion pattern for the user.

The terminal uses, as input, the animation object and the current orientation and position of the terminal in real space (for example, from built-in head-tracking sensors).

The terminal computes the relative position and orientation at which the avatar should appear in the user's field of view, such as in front of the user or overlaid on the user's own body.

The terminal advances the animation according to a local clock, applies each frame's joint poses to the avatar skeleton, and drives the graphics processing unit to draw the resulting frames on the display panel.

The terminal outputs an augmented-reality or virtual-reality visual that shows the optimal motion pattern in real time.

### Step 15:

The user observes and imitates the displayed motion.

The user receives, as input, the visual presentation of the three-dimensional avatar performing the optimal motion pattern, and optionally reads or listens to textual guidance displayed by the terminal.

The user adjusts body posture and movement to match the avatar's motion as closely as possible, for example by bending the knees more deeply or maintaining a straighter spine.

The user continues to perform the task while watching the avatar's movement and using the presented pattern as a real-time reference.

The user outputs updated physical motion, which is again captured by the information acquisition apparatus.

### Step 16:

The terminal captures synchronization cues and user interaction, if any.

The terminal uses, as input, local sensor readings such as head orientation, gaze direction, or user input via buttons or gestures.

The terminal can send timing information, such as the current phase of the animation, back to the server to help align updated analysis with the visual guidance.

The terminal may allow the user to trigger events, for example, by pausing or repeating certain segments of the animation, and forwards such commands to the server.

The terminal outputs control signals and timing data that influence subsequent processing on the server.

### Step 17:

The server acquires updated motion information for feedback.

The server takes, as input, new raw sensor data from the information acquisition apparatus while the user attempts to follow the optimal motion pattern.

The server processes these data as in Steps 1 and 2, computing updated joint angles, velocities, and other features, and compares them with the optimal motion pattern to compute deviation metrics such as absolute angle differences or timing offsets.

The server determines whether the user's motion has converged toward the optimal pattern or whether significant deviations persist.

The server outputs updated feature tensors and deviation metrics that will be used to refine prompts and, if necessary, request a new or adjusted optimal motion pattern from the generative AI model.

### Step 18:

The server generates a prompt sentence for motion guidance text.

The server uses, as input, the deviation metrics, the body capability profile, the current task content information, and the existing optimal motion pattern.

The server summarizes the main issues (for example, “excessive lumbar flexion at frame 20-40” or “insufficient knee flexion during lift initiation”) and encodes them into a natural-language prompt sentence, such as:

“The worker continues to flex the lower back about 10 degrees more than the recommended pattern during the first half of the lift, and knee flexion remains below 30 degrees. Provide concise, step-by-step guidance to increase knee flexion and reduce lower-back flexion, and suggest simple cues the worker can remember.”

The server outputs this prompt sentence and any structured context data required by the generative AI model.

### Step 19:

The server requests motion guidance text from the generative AI model.

The server takes, as input, the guidance-related prompt sentence constructed in Step 18.

The server transmits the tokenized prompt sentence to the generative AI model and specifies that the output format should be succinct instructions or bullet-like steps.

The server receives, as output, a motion guidance text containing detailed but concise instructions, warnings, or tips tailored to the user's current deviations from the optimal pattern.

### Step 20:

The server prepares and sends the motion guidance text to the terminal.

The server uses, as input, the motion guidance text produced by the generative AI model.

The server may segment the text into sentences or short paragraphs, tag each segment with an associated motion phase or animation frame range, and format the text according to the terminal's display constraints.

The server sends the formatted guidance text and its associated metadata to the terminal via the network interface.

The server outputs guidance data that the terminal can overlay alongside the three-dimensional animation.

### Step 21:

The terminal displays motion guidance text synchronized with the animation.

The terminal uses, as input, the guidance data received from the server and the current animation playback state.

The terminal displays the text near the avatar or at a fixed position in the user's field of view, updating the displayed segment as the animation progresses through its phases.

The terminal may additionally use a text-to-speech engine to convert the text into audio and output the audio through a speaker or headset while the animation is playing.

The terminal outputs synchronized multi-modal feedback that helps the user understand and correct motion in real time.

### Step 22:

The user refines motion based on updated visual and textual feedback.

The user uses, as input, the improved visualization of the optimal pattern and the accompanying motion guidance text or audio.

The user makes finer adjustments, such as modifying the timing of knee bending or the angle of the torso, and repeats the task while observing the changes in the avatar and listening to the instructions.

The user iteratively reduces deviations from the optimal motion pattern as indicated by the feedback.

The user outputs further refined motion, which is again captured, analyzed, and used to update the guidance in a continuing feedback loop.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

Conventional computer-implemented training support systems that analyze motion and physiological data suffer from several technical limitations at the level of data processing architecture, model coordination, and output control.

First, many existing systems merely capture raw image data and biological signal data and perform simple statistical calculations or threshold checks. Such systems do not systematically transform heterogeneous time-series data (video frames and biological signals) into synchronized, structured feature information that is suitable as input to multiple machine learning models. As a result, the processing burden on downstream components is increased, the reproducibility of analysis is reduced, and the overall computational pipeline becomes inefficient and difficult to scale.

Second, conventional systems typically treat motion analysis and advice generation as separate, loosely coupled processes. Motion feature extraction, risk estimation, and natural language advice generation are often executed by independent modules with ad hoc input formats. This separation leads to duplication of computations, redundant data transformations, and latency in generating results. It also makes it difficult for the system to adaptively modify advice generation based on the internal state of the analysis pipeline, resulting in suboptimal use of computing resources and model capabilities.

Third, while generative AI models are capable of producing detailed natural-language guidance, conventional systems generally provide such models with only coarse or unstructured prompts. Without a structured prompt that reflects the full set of computed features, index information, and model estimates, the generative AI model cannot effectively leverage the available analysis results. This causes unnecessary iterations between the analysis system and the generative AI model, increases network traffic and computational cost, and leads to inconsistent or unreliable advice outputs.

Fourth, many known systems do not tightly integrate the generation of visual representations, such as three-dimensional motion models, with the underlying numerical analysis. Visual content is often created manually or by separate rendering tools that are not synchronized with computed joint angles, center-of-gravity trajectories, or landing mechanics indices. This disconnect reduces the technical value of the generated visualizations for evaluation and feedback, and it prevents the system from programmatically emphasizing the specific motion parameters that are critical for performance improvement and injury risk reduction. Accordingly, there is a need for a computer-implemented system that: (i) acquires and synchronizes time-series image information and biological signal information with subject attributes to generate structured analysis data; (ii) executes posture estimation and feature computation in a unified processing flow to derive capability and load index information; (iii) integrates a trained machine learning model to estimate performance and injury risk indices; (iv) automatically structures these analysis results into a natural-language representation and constructs a prompt sentence that efficiently conditions a generative AI model; and (v) programmatically generates subject-specific training content, motion improvement information, and synchronized three-dimensional motion models or visual display information. Such a system should improve the efficiency, consistency, and technical reliability of the end-to-end computational pipeline from data acquisition to individualized advice output.

The specific processing by the specific processing unit 290 of the data processing device 12 in Example 2 is realized by the following means.

The present invention provides a server comprising a processor and a memory storing instructions which, when executed by the processor, cause the server to acquire, for a subject, time-series image information from an imaging device and biological signal information from a biological measurement device, to associate the time-series image information and the biological signal information with measurement time information and subject attribute information to generate analysis data, to execute posture estimation processing on the time-series image information included in the analysis data so as to extract time-series posture information indicating positions of body parts of the subject, to compute feature information including at least joint angle information, movement speed information, motion phase information, and biological response information based on the time-series posture information and the biological signal information, to derive capability index information and load index information by comparing the feature information with reference feature information of professional exercise performers, to input the feature information, the capability index information, and the load index information into a machine learning model trained in advance on reference data and to estimate, by using the machine learning model, performance index information, injury risk index information, and motion classification information for the subject, to structure analysis result information including at least the subject attribute information, the feature information, the capability index information, the load index information, and an estimation result of the machine learning model into a natural-language description, to generate a prompt sentence including an instruction statement according to a content of the analysis result information and a training objective of the subject, to perform generation processing of instructing a generative AI model, by the prompt sentence, to generate subject-specific training content and motion improvement information, and to output the training content and the motion improvement information received from the generative AI model as advice information associated with numerical index information and, when necessary, to associate and present the advice information with a three-dimensional motion model or visual display information. This enables improvement of the overall computer system performance by providing an integrated, machine-oriented data processing pipeline that converts heterogeneous motion and biological data into structured feature and index information, optimally conditions a generative AI model through a programmatically constructed prompt sentence, and consistently generates synchronized textual and visual outputs, thereby reducing computational redundancy, latency, and inconsistency in motion analysis and individualized training advice generation.

The term “subject” refers to a human individual whose motion and biological signals are acquired, analyzed, and evaluated by the system.

The term “imaging device” refers to an electronic apparatus that captures time-series image information of a subject, such as a camera or an image sensor, and outputs the captured information in a digital format.

The term “biological measurement device” refers to an electronic apparatus that measures biological signal information of a subject, such as a heart rate sensor, an electrocardiogram sensor, or another physiological sensor, and outputs the measured information in a digital format.

The term “time-series image information” refers to image data representing successive images of a subject over time, including video data or sequential still-image data each associated with a time point.

The term “biological signal information” refers to time-series data representing physiological activity of a subject, including heart rate, bioelectrical signals, or other measurable biological parameters associated with time.

The term “measurement time information” refers to information indicating a time at which the time-series image information and the biological signal information are acquired, and is used to temporally align different data streams.

The term “subject attribute information” refers to information that characterizes a subject, including at least age, biological sex, physical characteristics, exercise history, or training objectives.

The term “analysis data” refers to a data structure that includes the time-series image information, the biological signal information, the measurement time information, and the subject attribute information, organized for subsequent computational processing.

The term “posture estimation processing” refers to a computational procedure that analyzes the time-series image information to estimate positions or orientations of body parts of a subject in each frame, thereby generating posture information.

The term “time-series posture information” refers to data representing estimated positions or orientations of multiple body parts of a subject at successive time points, typically expressed as coordinates or angles.

The term “feature information” refers to numerical or categorical data derived from the time-series posture information and the biological signal information, including joint angles, movement speeds, motion phases, and biological responses.

The term “joint angle information” refers to feature information that quantifies angles formed by segments of a subject's body, such as angles at knees, hips, ankles, or other joints, over time.

The term “movement speed information” refers to feature information that quantifies velocities or speeds of body parts or the center of mass of a subject, computed from changes in positions over time.

The term “motion phase information” refers to feature information that segments a motion into distinct phases, such as preparation, take-off, flight, and landing, determined based on posture or kinematic patterns.

The term “biological response information” refers to feature information that characterizes physiological responses of a subject to motion, including changes in heart rate, variability, or other physiological indices over time.

The term “reference feature information” refers to feature information that has been computed from motion data and biological signal data of reference individuals, such as professional exercise performers, and used as a basis for comparison.

The term “capability index information” refers to one or more numerical or categorical indices that represent a subject's physical capability or performance level, calculated by comparing the subject's feature information with reference feature information.

The term “load index information” refers to one or more numerical or categorical indices that represent a subject's physical load or stress level, calculated by comparing the subject's feature information and biological responses with reference feature information.

The term “machine learning model” refers to a computational model that has been trained on reference data to learn a mapping from input feature information to output indices or classifications, and that performs estimation or prediction when provided with new input data.

The term “performance index information” refers to one or more indices output by the machine learning model that quantify a subject's motion performance, such as efficiency, potential, or quality of movement.

The term “injury risk index information” refers to one or more indices output by the machine learning model that quantify a subject's potential risk of injury, such as likelihood of overload or unsafe movement patterns.

The term “motion classification information” refers to categorical information output by the machine learning model that classifies a subject's motion into one or more predefined categories, such as particular movement patterns or technique types.

The term “analysis result information” refers to an aggregated set of data that includes the subject attribute information, the feature information, the capability index information, the load index information, and outputs of the machine learning model.

The term “natural-language description” refers to a text-based representation of the analysis result information expressed in a human-readable language, such as sentences or paragraphs.

The term “prompt sentence” refers to a text input provided to a generative AI model, including an instruction statement and contextual information, which conditions the generative AI model to generate a desired output.

The term “instruction statement” refers to a portion of the prompt sentence that explicitly describes a requested task or output, such as generation of training content or motion improvement information for a subject.

The term “generative AI model” refers to a trained computational model that generates new data, including text or other content, in response to an input prompt sentence, based on patterns learned from training data.

The term “training content” refers to information describing an exercise or training plan for a subject, including recommended activities, intensities, frequencies, or progressions.

The term “motion improvement information” refers to information describing modifications or adjustments to a subject's motion, including technique changes, posture corrections, or specific focus points to improve performance or reduce injury risk.

The term “advice information” refers to an output that includes at least the training content and the motion improvement information, optionally accompanied by numerical indices or visual elements.

The term “numerical index information” refers to numerical values, such as scores, ratings, or indices, that are associated with the advice information and indicate performance levels, risks, or priorities.

The term “three-dimensional motion model” refers to a computer-generated representation of a subject's motion in three-dimensional space, constructed from posture information and motion patterns, and suitable for visual display.

The term “visual display information” refers to image or graphical information, including two-dimensional or three-dimensional representations, charts, or overlays, used to visually present motion, indices, or advice information to a user.

In one embodiment, a server, a terminal, and a user cooperate to implement the invention.

The server includes at least one processor, a memory, a communication interface, and a storage device. The storage device stores executable instructions, trained model parameters, and reference data. The server executes an operating system and application software implemented, for example, using a general-purpose programming language and a numerical computation library. The server may use a graphics processing unit to accelerate pose estimation and neural network inference.

The terminal includes a processor, a memory, a communication interface, and an input/output interface. The terminal executes an application that communicates with the server, controls an imaging device, and controls a biological measurement device. The terminal may be implemented as a portable information processing device or a stationary information processing device.

The user sets up an imaging device and a biological measurement device in a physical environment. The imaging device captures time-series image information of a subject who performs physical movements. The biological measurement device measures biological signal information, such as heart rate, synchronized with the physical movements. The user positions the devices to cover the subject's full body and configures the terminal to connect to the imaging device and the biological measurement device.

The terminal acquires digital video data from the imaging device using a camera application programming interface or a video capture library. The terminal acquires digital biological signal data from the biological measurement device using a wireless communication protocol or a wired protocol. The terminal attaches measurement time information and subject attribute information, including at least age, sport type, and training objective, to the acquired data. The terminal then transmits a structured dataset to the server using a network protocol.

The server stores the received video data and biological signal data in an analysis data structure. The analysis data structure includes fields for time-series image information, time-series biological signal information, measurement time information, subject attribute information, and identifiers for subsequent processing. By standardizing the analysis data structure, the server reduces repeated parsing, simplifies indexing, and improves cache locality, thereby improving internal data management and computational efficiency.

The server executes posture estimation processing on the time-series image information. In one embodiment, the server uses a pose-estimation library to detect body keypoints in each frame of the video. The server may represent the detected keypoints as a matrix whose rows correspond to time steps and whose columns correspond to coordinate components of body parts. The server stores this matrix as time-series posture information.

The server computes feature information from the time-series posture information and the biological signal information. The server calculates joint angles by applying trigonometric functions to triplets of keypoints corresponding to joints. The server calculates movement speeds by computing finite differences of positions over time. The server identifies motion phases by applying rule-based segmentation conditions on vertical velocity, joint angle trajectories, and ground contact indicators. The server also aggregates biological responses over motion phases, for example, by computing heart rate increase between start and end of a motion set. The server stores the resulting feature information in a feature vector or a feature sequence associated with each analysis session.

The server compares the feature information with reference feature information derived from professional exercise performers. The server stores the reference feature information in a database as statistics, such as means, variances, and percentile thresholds for each feature dimension. The server computes capability index information and load index information by calculating standardized scores or distances between the subject's feature vector and the reference statistics. This comparison is executed programmatically and automatically, according to predetermined mathematical formulas, and not by manual assessment.

The server then inputs the feature information, the capability index information, and the load index information into a machine learning model. In one embodiment, the machine learning model is implemented as a neural network executed by a numerical computation framework. The neural network may be a feedforward network or a recurrent network, with multiple layers including at least an input layer, one or more hidden layers, and an output layer. Each hidden layer applies a linear transformation followed by a non-linear activation function such as a rectified linear unit. The server normalizes the input feature information using parameters derived from the training dataset, in order to stabilize training and inference.

The server trains the machine learning model in an offline training phase using reference data from multiple professional exercise performers and other reference subjects. During training, the server divides the reference data into batches, computes a loss function, such as a mean squared error for continuous indices and a cross-entropy loss for classification outputs, and updates model weights using a gradient-based optimization algorithm such as stochastic gradient descent or an adaptive method. The server may apply regularization techniques such as weight decay and dropout, and may apply data augmentation techniques such as adding small random perturbations to posture information to improve robustness. The server stores the learned weights and bias parameters in the storage device.

The server uses the trained machine learning model in an online inference phase to estimate performance index information, injury risk index information, and motion classification information. Because the server uses a fixed, optimized model and pre-computed normalization parameters, the server reduces inference latency and improves prediction consistency compared to ad hoc rule-based scoring.

The server aggregates the subject attribute information, the feature information, the capability index information, the load index information, and the estimation result of the machine learning model into analysis result information. The server then structures the analysis result information into a natural-language description. This structuring step uses a deterministic template-based algorithm that maps numerical indices and classification labels into text segments. For example, the server may generate statements such as “Maximum knee flexion angle is below the reference mean by a predetermined threshold” or “Predicted injury risk index is within a specified range.” This deterministic pre-processing reduces the burden on subsequent generative processing and ensures that key technical metrics are explicitly present in the prompt.

The server generates a prompt sentence for a generative AI model. The prompt sentence includes a description of the subject, a summary of the computed metrics, and an instruction statement specifying the desired output. For example, the server may construct a prompt sentence as follows:

“You are a strength and conditioning coach. The subject is a 17-year-old male basketball player whose goal is to increase vertical jump height. Motion-capture and heart-rate analysis show: Current vertical jump height: 45 cm. Maximum knee flexion angle before take-off: 70 degrees, which is about 20 degrees less than professional reference (90 degrees). Movement pattern: insufficient hip drive and early knee extension. Landing impact index: relatively high, indicating increased knee stress. Heart rate increase during the jump set: moderate, suggesting some fatigue with repeated jumps. Using this information, create a detailed 6-week training plan to improve vertical jump while reducing knee injury risk. Specify warm-up routines, strength exercises, plyometric drills, frequency and intensity per week, and key technical coaching cues. Provide concise, actionable recommendations in bullet points.” The server sends the constructed prompt sentence to a generative AI model executed on the same server or on a separate computing resource. The generative AI model is a trained text generation model that maps the prompt sentence into textual outputs according to parameters learned from a large corpus. By providing a prompt sentence that is tightly constrained and enriched with structured metrics, the server reduces the size and ambiguity of the model's search space, which in turn improves generation speed and output consistency. This behavior differs from manual preparation of free-form prompts, and constitutes a computer-implemented method of efficiently conditioning the generative AI model.

The server receives the generated training content and motion improvement information from the generative AI model. The server associates this advice information with the numerical index information and, when necessary, links it to a three-dimensional motion model or visual display information. The server may construct a three-dimensional motion model by mapping the posture information and recommended motion patterns into a three-dimensional coordinate system and rendering key frames or animated sequences. The server emphasizes joint angle changes, center-of-gravity trajectories, and landing mechanics that are directly related to the computed indices. By programmatically emphasizing these aspects, the server provides a visualization that is directly synchronized with the internal analysis results, improving technical interpretability.

The terminal receives the advice information and visualization data from the server and displays them on a screen. The terminal may show numerical indices, text-based training plans, and three-dimensional animations on a unified user interface. The user observes the displayed information and provides physical training guidance to the subject in a real-world environment. The user may adjust the placement of the imaging device and the biological measurement device, repeat measurements, and confirm improvements by comparing new computed indices with previous results.

This configuration provides several technical effects. Because the server structures raw heterogeneous sensor data into a normalized analysis data structure, and because the server integrates pose estimation, feature computation, index calculation, model inference, deterministic natural-language structuring, and prompt generation in a unified pipeline, the server reduces redundant computations and repeated data transformations. This yields improved processing speed and reduced memory overhead. The use of a pre-trained neural network, optimized for the specific type of motion data and features, improves estimation accuracy relative to simple threshold-based rules. The explicit comparison to reference feature information and the calculation of capability and load indices allow consistent scaling across subjects and sessions, which improves the stability of downstream generative processing.

The server's deterministic pre-processing of analysis results and programmatic construction of a prompt sentence improve communication with the generative AI model. This design reduces the amount of data transmitted and the number of interaction rounds required to obtain a usable training plan, thereby reducing communication load and total processing time. Furthermore, by embedding structured indices and labels into the prompt sentence, the server ensures that the generative AI model considers key technical parameters in a consistent manner, which improves the relevance and precision of generated advice.

The described implementation does not merely automate a human mental process. Human experts cannot manually synchronize high-frequency video and biological signal streams, compute multi-dimensional kinematic features, and perform high-dimensional optimization-based inference at comparable speed and scale. The server applies specific numerical algorithms, data structures, and learned parameter sets that are impractical to execute manually. The integration of these components in a particular sequence and format yields measurable improvements in processing throughput, estimation accuracy, and network efficiency.

In alternative embodiments, the server may use different neural network architectures. For example, the server may employ a temporal convolutional network or a recurrent neural network to process sequences of feature vectors. The server may also employ an attention-based mechanism to weight time steps or joints that are more relevant to injury risk. The server may vary the loss functions, such as using a combination of regression loss and ranking loss, or may adopt different optimization algorithms. The pose estimation component may be implemented using a different algorithm, as long as it outputs time-series posture information representing body part positions.

In another embodiment, the terminal performs at least part of the feature computation, such as initial pose estimation or basic joint angle calculation, and transmits intermediate feature information rather than raw video to the server. This reduces network bandwidth usage and further decreases communication load. The server then performs index calculation, model inference, prompt generation, and advice association as described above. In yet another embodiment, the system uses separate servers for pose estimation, model inference, and generative processing, with a message-based data flow between modules, while preserving the core operations and data structures.

The invention can also be applied to other movement types and other measurement devices. For example, the imaging device may be a depth sensor, and the biological measurement device may be an electromyography sensor. The same processing framework, including structured analysis data, feature computation, index calculation, neural network inference, and prompt-based generative advice, can be used with appropriate modifications to feature definitions and reference datasets. In each case, the server provides a technical improvement by transforming complex, heterogeneous sensor streams into a structured, machine-optimized representation, and by controlling both analytic and generative components through specific data formats and algorithms, thereby achieving higher efficiency and reliability than conventional, loosely coupled systems.

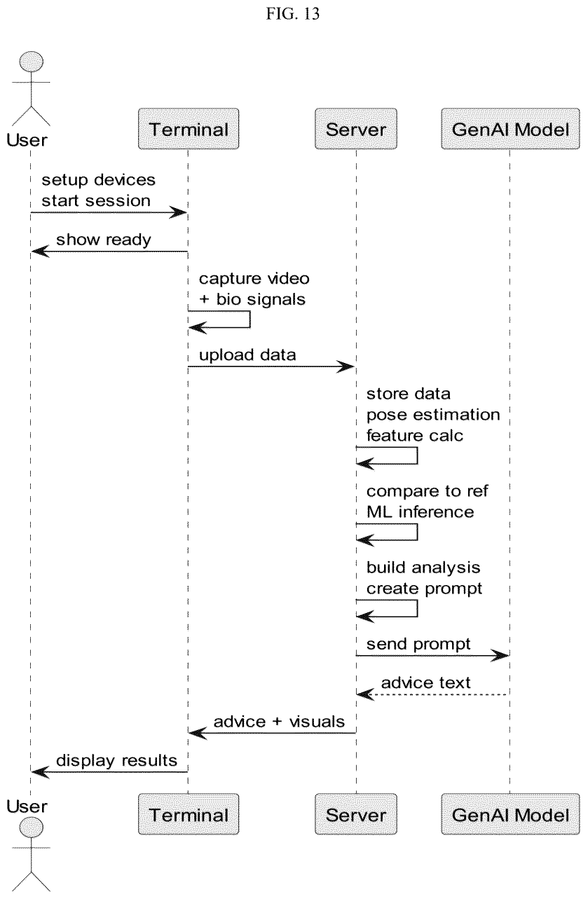

The following describes the processing flow using FIG. 13.

### Step 1:

The user configures the measurement environment.

The user places an imaging device in a position where the full body of the subject is visible during movement and attaches a biological measurement device to the subject.

Input: Physical environment, subject, imaging device, biological measurement device.

Output: A prepared measurement setup in which the devices are physically positioned and activated so that they can capture motion and biological signals.

The user powers on the devices, confirms that indicator lights or on-screen messages show that the devices are ready, and positions the subject within the field of view of the imaging device.

### Step 2:

The terminal establishes connections with the imaging device and the biological measurement device.

The terminal executes an application that discovers the devices via a communication interface, such as a wireless or wired protocol, and performs a pairing or connection procedure.

Input: Device identifiers, network configuration, user-selected subject profile.

Output: Active communication sessions between the terminal and each device, and device status data indicating that streaming is available.

The terminal sends control commands to the imaging device to start preview mode and to the biological measurement device to start transmitting current measurements, and displays connection status icons to the user.

### Step 3:

The terminal acquires time-series image information and biological signal information. The terminal captures video frames from the imaging device using an application programming interface and reads biological signal samples from the biological measurement device in real time.

Input: Raw sensor streams from the imaging device and the biological measurement device.

Output: A buffered sequence of time-stamped video frames and a buffered sequence of time-stamped biological samples stored in the terminal memory.

The terminal attaches measurement time information from its system clock to each frame and each biological sample, and temporarily stores these in a structured buffer so that later processing can match them by time.

### Step 4:

The terminal associates the captured data with subject attribute information and creates a transmission payload.

The terminal retrieves subject attribute information, such as age, sport type, and training goal, from a local profile database or user input and bundles it with the recorded data.

Input: Buffered video frames, buffered biological samples, subject attribute information, measurement time information.

Output: A structured payload containing time-series image information, time-series biological signal information, time stamps, and subject attributes formatted for network transmission.

The terminal compresses the video into a file, serializes the biological samples into a file or data stream, and organizes a metadata record that references both, preparing a single request to the server.

### Step 5:

The terminal transmits the structured payload to the server.

The terminal initiates a network request to the server using a communication protocol and sends both the data files and associated metadata.

Input: Structured payload that includes the recorded data and metadata.

Output: A completed data upload to the server and a server-side acknowledgment received by the terminal.

The terminal monitors the upload progress, retries segments if necessary, and, after receiving a successful response code from the server, records a local log entry for the completed session.

### Step 6:

The server receives and stores the uploaded data as analysis data.

The server accepts the network request, validates file integrity, and assigns a unique session identifier to the uploaded data.

Input: Structured payload sent by the terminal.

Output: Analysis data stored in the server's storage subsystem and a session identifier stored in a database.

The server writes the video file and biological signal file to persistent storage locations and creates a database record that references these file paths and the subject attribute information.

### Step 7:

The server performs posture estimation on the time-series image information.

The server loads the video file corresponding to the session identifier and processes each frame using a pose-estimation component to extract positions of body parts.

Input: Time-series image information from the analysis data.

Output: Time-series posture information representing body part positions and orientations for each frame.

The server decodes each frame, runs the pose-estimation algorithm, obtains coordinates of key body joints, and stores them in a time-aligned array or matrix structure indexed by frame number and joint index.

### Step 8:

The server synchronizes the time-series posture information with the biological signal information.

The server loads the biological signal file, interpolates or samples the data to align it with the timestamps of the posture information, and constructs unified time-series records.

Input: Time-series posture information, time-series biological signal information, measurement time information.

Output: Synchronized sequences where each posture record is associated with a corresponding biological sample or derived biological measure.

The server creates a combined data structure in which each time step includes joint coordinates or angles and a corresponding biological value, such as heart rate, enabling joint analysis across both modalities.

### Step 9:

The server computes feature information from the synchronized sequences.

The server transforms the time-series posture information into joint angles, movement speeds, and motion phases, and aggregates biological responses for each phase.

Input: Synchronized posture and biological sequences.

Output: Feature information that includes joint angle information, movement speed information, motion phase information, and biological response information for the session.

The server calculates joint angles through vector operations between body part positions, computes velocities by differentiating position sequences, detects transitions between phases, such as preparation, take-off, and landing, and summarizes changes in biological signals, such as heart rate increase during each detected phase.

### Step 10:

The server derives capability index information and load index information by comparing the feature information to reference feature information.

The server retrieves reference statistics, such as mean and variance for each feature dimension, from a database of reference subjects, including professional performers, and computes standardized measures.

Input: Feature information for the subject, reference feature information from the database.

Output: Capability index information and load index information that quantitatively describe the subject's relative performance and physical stress level.

The server computes difference values, standardized scores, or percentile rankings for each relevant feature, aggregates these values into indices, and stores these indices with the session identifier.

### Step 11:

The server estimates performance index information, injury risk index information, and motion classification information using a machine learning model.

The server loads a trained neural network model, normalizes the feature information and indices, and performs forward propagation through the network to generate estimates.

Input: Feature information, capability index information, load index information.

Output: Estimated performance index information, injury risk index information, and motion classification information for the subject.

The server arranges the inputs into a numeric vector, scales each feature according to stored normalization parameters, passes the vector through the network layers, and reads outputs from the final layer representing the indices and class labels.

### Step 12:

The server constructs analysis result information from all computed data.

The server aggregates the subject attribute information, the feature information, the capability index information, the load index information, and the model estimation results into a single structured representation.

Input: Subject attribute information, feature information, capability index information, load index information, model outputs.

Output: Analysis result information that encapsulates the entire numerical evaluation of the session.

The server creates a record or object that groups all related values under the session identifier, ensuring that subsequent processing can access the data in a single query or function call.

### Step 13:

The server converts the analysis result information into a natural-language description.

The server applies a template-based text generation procedure that maps numeric values and classification labels to descriptive phrases and sentences.

Input: Analysis result information with numeric and categorical values.

Output: A natural-language description that explains the subject's performance, weaknesses, and risk factors.

The server inserts values, such as joint angles and indices, into pre-defined sentence templates, rounds numeric values to specified precision, and generates coherent text paragraphs summarizing the key findings.

### Step 14:

The server generates a prompt sentence for a generative AI model based on the natural-language description and training objectives.

The server combines the descriptive summary with an instruction statement that specifies the desired type of output, such as a structured training plan or motion corrections.

Input: Natural-language description, subject's training objectives included in subject attribute information.

Output: A prompt sentence that conditions the generative AI model to produce subject-specific training content and motion improvement information.

The server appends explicit instructions to the descriptive text, such as required duration of the plan, types of exercises, and format of the output, and ensures that all relevant indices and findings are included in the prompt sentence.

### Step 15:

The server transmits the prompt sentence to the generative AI model and obtains generated advice.

The server sends the prompt sentence through an application programming interface to the generative AI model and receives textual responses describing recommended training content and motion improvement methods.

Input: Prompt sentence constructed from the analysis results.

Output: Generated advice text containing training content and motion improvement information tailored to the subject.

The server waits for the generative AI model to complete its generation, parses the returned text, and verifies that the output conforms to expected content categories, such as warm-up, main exercises, and technical cues.

### Step 16:

The server associates the generated advice with numerical indices and visual representations.

The server links portions of the generated advice text to specific capability indices, load indices, and motion classification results, and, when necessary, prepares parameters for generating a three-dimensional motion model or other visual display information.

Input: Generated advice text, analysis result information including numeric indices and posture data.

Output: Advice information that consists of the advice text, associated indices, and parameters for visual displays.

The server marks which part of the text corresponds to improvement of which index, generates annotations for visual elements, and prepares a representation that can drive three-dimensional rendering of key movements.

### Step 17:

The server sends the advice information and associated visual data to the terminal.

The server packages the advice text, indices, and visual parameters into a response structure and transmits it to the terminal via the communication interface.

Input: Advice information and visual parameters generated on the server.

Output: A response message received by the terminal that contains all data needed for presentation to the user.

The server attaches the session identifier to the response, logs the time and size of the transmission, and ensures that any large visual assets are referenced via identifiers or links to reduce redundant transfers.

### Step 18:

The terminal presents the advice information and visual displays to the user.

The terminal interprets the received response, renders the numerical indices in charts or tables, displays the advice text, and optionally renders three-dimensional motion models or annotated images.

Input: Response message from the server containing advice text, indices, and visual parameters.

Output: A user interface display that allows the user to review the analysis and training recommendations.

The terminal draws graphs that show changes in key metrics, highlights high-risk indices, presents the training plan in readable sections, and plays animations that visually emphasize recommended joint angles and movements.

### Step 19:

The user applies the advice to physical training and optionally initiates new sessions for reassessment.

The user reads the displayed recommendations, instructs the subject in performing specific exercises and motion corrections, and may schedule future measurements to track progress.

Input: Displayed advice information, real-world training environment.

Output: Adjusted training routines for the subject and, when reassessment is performed, new measurement sessions that produce additional data.

The user observes changes in the subject's performance and comfort, uses the terminal to start follow-up measurements, and relies on the system to compute updated indices and generate revised advice based on the new data.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

Conventional motion-analysis and coaching systems mainly log sensor and video data and then apply fixed rule-based logic or static machine-learning models to evaluate a subject's motion. These systems typically operate as offline analysis pipelines and are not designed to dynamically adapt their internal behavior based on high-level, human-readable instructions. As a result, the systems are limited in their ability to flexibly change optimization objectives, such as prioritizing safety over performance, or adapting to different users, tasks, or environmental conditions without redesigning models or rewriting program code.

Furthermore, known systems generally treat numerical sensor streams and any associated textual guidance as separate, loosely coupled components. The computational pipeline lacks an integrated mechanism for using natural-language instructions to steer model behavior at runtime. This separation hinders the efficient reuse of a common generative model across diverse application contexts, such as sports training, industrial work support, and machine or robot control, and makes it difficult to reconfigure the system in response to user feedback or changing operating conditions.

Additionally, many existing systems focus solely on discriminative evaluation of motion quality and do not provide a robust framework for generating new, optimized motion patterns. Even when some form of motion generation is used, it is often specialized for a single task, manually tuned, and not configured to systematically evaluate multiple candidate motions against multiple objective measures such as energy consumption, joint load, required time, and safety. Consequently, the systems are unable to automatically propose and refine motion patterns that balance performance and risk according to context-dependent requirements.

Moreover, conventional motion-optimization systems generally do not incorporate real-time biological state and emotional state into the control of a generative model. The computational pipeline therefore fails to adapt its optimization policy when indicators such as fatigue, stress, or anxiety are detected. As a result, generated recommendations can be misaligned with the subject's current physical and psychological condition, leading to sub-optimal guidance and potentially increased risk of injury or error.

In summary, there is a need for an improved computer-implemented system that: (i) tightly couples numerical motion and physiological data with prompt-driven control of a generative AI model; (ii) dynamically generates and updates prompt sentences in response to real-time biological state, emotional state, task type, and environmental context; (iii) efficiently generates and evaluates multiple candidate motion patterns according to multiple objective indices; and (iv) outputs optimized motion patterns both as three-dimensional visual guidance for human users and as control signals for machines or work equipment. Such a system would address deficiencies in flexibility, adaptability, and optimization capability found in prior computer-implemented motion-analysis and motion-generation techniques, thereby improving the overall functioning of the motion-optimization computer platform itself.

The specific processing by the specific processing unit 290 of the data processing device 12 in Application Example 2 is realized by the following means.

The present invention provides a server comprising a processor configured to execute machine instructions that (i) perform a learning process on time-series data by using a prompt sentence that instructs a generative AI model to learn attributes related to a body and information related to physical motion, (ii) acquire, by using a detection device and an imaging device, biological state information and physical motion information of a target subject, extract, from the acquired information, feature values including joint positions, joint angles, motion velocities, and biological signals, and store the feature values in a data structure accessible to the generative AI model, (iii) input the extracted feature values and the prompt sentence to the generative AI model and execute a generation process in which the generative AI model generates a motion pattern that allows the target subject to satisfy a predetermined performance index while reducing load and damage risk, (iv) generate three-dimensional video data that visualizes the motion pattern by deforming three-dimensional shape data having a skeletal structure along a time axis on the basis of the motion pattern generated by the generative AI model, (v) transmit the three-dimensional video data to an information presentation device and present the three-dimensional video data as instruction information for improvement of motion of the target subject or of a machine apparatus, (vi) dynamically generate or update the prompt sentence to be input to the generative AI model in accordance with at least one of a biological state, an emotional state, a task type, an environmental condition, and an evaluation index related to the target subject, and (vii) cause the generative AI model to generate a plurality of candidate motion patterns, evaluate the plurality of candidate motion patterns on the basis of at least one of energy consumption, joint load, required time, and a safety index, select an optimal motion pattern from among the plurality of candidate motion patterns, and convert the optimal motion pattern into a control signal for a machine apparatus or a work machine so as to adjust operation of the machine apparatus or the work machine. This enables the underlying computer system to computationally integrate sensor-derived motion and biological data with dynamically generated prompt sentences, to control a generative AI model in real time, to algorithmically search and select motion patterns that satisfy context-dependent objectives, and to output both adaptive visual guidance and machine control signals, thereby improving the flexibility, adaptability, and technical performance of computer-implemented motion optimization.

The term “system” refers to a combination of hardware and software components, including at least one processor, memory, input devices, output devices, and communication interfaces, that cooperatively execute instructions to perform functions described in the claims.

The term “processor” refers to one or more hardware processing units, such as a central processing unit, a graphics processing unit, a digital signal processor, or any combination thereof, configured to execute machine-readable instructions.

The term “time-series data” refers to data represented as sequences of values indexed by time, such as sequences of joint angles, positions, velocities, accelerations, or biological signals sampled at discrete time intervals.

The term “prompt sentence” refers to a text string that encodes a human-readable instruction or description and is provided as input to a generative AI model in order to influence or control the behavior or output of the generative AI model.

The term “generative AI model” refers to a machine-learned model configured to generate new data samples, such as motion patterns, based on input information including feature values and at least one prompt sentence.

The term “attributes related to a body” refers to physical characteristics of a subject, including but not limited to body size, limb length, strength, flexibility, and other biomechanical properties.

The term “information related to physical motion” refers to data describing movement of a subject or a machine, including but not limited to joint positions, joint angles, trajectories, velocities, accelerations, and temporal patterns of such movements.

The term “target subject” refers to a physical entity whose motion is analyzed or optimized, including a human individual, an animal, a robot, or a mechanical apparatus.

The term “biological state information” refers to data indicating a physiological condition of a target subject, including but not limited to heart rate, respiration rate, muscle activity, body temperature, or other measurable biological signals.

The term “physical motion information” refers to measurement data that directly or indirectly represents movement of a target subject, such as kinematic data obtained from sensors or imaging devices.

The term “detection device” refers to a hardware device configured to sense physical quantities, including but not limited to motion sensors, inertial sensors, force sensors, position sensors, or physiological sensors.

The term “imaging device” refers to a hardware device configured to capture image data or video data, such as a camera, depth sensor, or other optical imaging apparatus.

The term “feature values” refers to numerical values derived from raw sensor or image data that quantitatively represent aspects of motion or biological state, including but not limited to joint positions, joint angles, velocities, accelerations, and processed biological signals.

The term “joint position” refers to a spatial coordinate representing the location of a joint or articulation point of a body or mechanism in one, two, or three dimensions.

The term “joint angle” refers to a rotational measure representing the relative orientation between two segments or links connected by a joint.

The term “motion velocity” refers to a rate of change of position or joint angle of a body or mechanism over time.

The term “biological signal” refers to a time-varying measurement originating from a physiological process of a target subject, including but not limited to cardiac, respiratory, muscular, or neural activity.

The term “learning process” refers to a computation in which a model's parameters are updated based on training data so that the model improves its ability to perform a task such as prediction or generation.

The term “generation process” refers to a computation in which a generative AI model produces output data, such as a motion pattern, based on one or more inputs including feature values and at least one prompt sentence.

The term “motion pattern” refers to a sequence of states representing movement over time, including but not limited to a sequence of joint positions, joint angles, and corresponding timing information.

The term “performance index” refers to a quantitative measure used to evaluate a motion pattern, including but not limited to task completion time, accuracy, efficiency, speed, power output, or a composite score derived from multiple parameters.

The term “load” refers to a physical burden or mechanical stress experienced by a body or mechanism, including but not limited to joint torque, muscle force, or structural stress.

The term “damage risk” refers to a probability or likelihood that a motion pattern may cause injury to a biological subject or failure in a mechanical component.

The term “three-dimensional video data” refers to data representing a time-varying three-dimensional visualization, including animated three-dimensional models or skeletons displayed over a sequence of frames.

The term “three-dimensional shape data” refers to digital data that defines a three-dimensional geometric representation of an object or body, including meshes, surfaces, volumes, or skeletal structures.

The term “skeletal structure” refers to an abstract or geometric representation of connected segments and joints that approximates the structure of a body or mechanism for purposes of motion representation and animation.

The term “time axis” refers to an ordered dimension representing progression of time, along which values such as joint positions or three-dimensional poses are defined.

The term “information presentation device” refers to any device configured to output information to a user, including but not limited to a display, a head-mounted display, a projector, a speaker, or a combination thereof.

The term “instruction information” refers to data presented to a user or system that indicates how to perform, modify, or control a motion, including visual, auditory, or haptic guidance.

The term “machine apparatus” refers to a mechanical system that performs tasks or operations, including but not limited to industrial machines, robots, or automated tools.

The term “work machine” refers to a machine apparatus used to perform work in an industrial, commercial, or service environment, such as a production line machine, assembly robot, or material handling equipment.

The term “emotional state” refers to a psychological or affective condition of a target subject, such as stress, anxiety, excitement, relaxation, or other emotional categories inferred from data.

The term “task type” refers to a classification of the activity being performed by the target subject or the machine apparatus, including but not limited to sports movement, industrial work, assembly operation, or maintenance operation.

The term “environmental condition” refers to one or more parameters related to the external environment in which the target subject or machine apparatus operates, such as temperature, noise level, lighting condition, or workspace layout.

The term “evaluation index” refers to a numerical or categorical metric used to assess quality or suitability of a motion pattern, including performance-related, safety-related, comfort-related, or efficiency-related measures.

The term “candidate motion pattern” refers to one of multiple motion patterns generated by the generative AI model for the purpose of being compared and evaluated prior to selecting an optimal motion pattern.

The term “energy consumption” refers to an amount of energy predicted or measured as required to execute a motion pattern, by a biological subject or by a machine apparatus.

The term “joint load” refers to mechanical stress, torque, or force acting on a joint within a body or mechanism during execution of a motion pattern.

The term “required time” refers to a duration taken to complete a defined motion or task according to a given motion pattern.

The term “safety index” refers to a metric that represents a degree of safety associated with a motion pattern, including a measure of likelihood of injury, collision, or malfunction.

The term “optimal motion pattern” refers to a motion pattern selected from multiple candidate motion patterns based on one or more evaluation indices as meeting or best satisfying predetermined criteria.

The term “control signal” refers to a signal, which may be digital or analog, transmitted to a machine apparatus or work machine to cause the machine apparatus or work machine to perform operations in accordance with a motion pattern.

The term “compare” refers to compute one or more differences, similarities, or other relational measures between two or more sets of data, such as generated motion data and current motion data.

The term “posture” refers to an arrangement or configuration of body segments or mechanical elements at a given time, as defined by joint positions and joint angles.

The term “motion trajectory” refers to a path of motion of one or more points or segments over time in one, two, or three dimensions.

The term “sequentially update” refers to modify data representing a state, such as posture or trajectory, in a stepwise manner over time based on new input or evaluation results.

The term “current motion data” refers to motion information acquired for the target subject at or near a present time during operation of the system.

The term “current biological state data” refers to biological state information acquired for the target subject at or near a present time during operation of the system.

In one embodiment, a server, a terminal, and a user cooperate to implement the claimed system. The server includes at least one processor, a memory storing machine-executable instructions, a communication interface, and non-transitory storage for motion and model data. The terminal includes an imaging device, one or more detection devices, a display device, and optionally a head-mounted display. The user is a human operator, athlete, worker, or supervisor who performs physical motions and interacts with the system.

Server executes a program stored in the memory to realize the functions described in the claims. Server uses a runtime environment such as an interpreted language engine and a numerical computation library to perform matrix operations, tensor operations, and optimization procedures. For example, server uses a numerical computing framework to implement a generative AI model as a neural network, and uses a multimedia library to process image and video streams. These specific software components run on general-purpose processors and, in some embodiments, on dedicated accelerators such as graphics processing units.

Server maintains structured data for each target subject. Server stores a subject profile including identifiers, anthropometric parameters (for example, limb lengths, body mass), historical performance indices, and health constraints. Server stores time-series data structures for motion and biological signals; each time-series entry contains a timestamp, a vector of joint positions and joint angles, a vector of motion velocities, and one or more biological signals such as heart rate or muscle activation levels. Server also stores prompt sentences in a text store, and stores model parameters of the generative AI model in a model repository.

Terminal acquires physical motion information and biological state information of the user. Terminal uses an imaging device, such as an RGB camera or depth camera, to capture images or video of the user's body. Terminal uses a detection device, such as an inertial measurement unit, a force sensor, or a heart-rate sensor, to capture corresponding sensor streams. Terminal tags each sensor packet and frame with a timestamp and transmits the streams to server through the communication interface. Terminal may apply pre-processing such as image resizing, region-of-interest cropping, and compression to reduce communication load and improve throughput.

Server receives the physical motion information and the biological state information from terminal. Server uses an image processing library, such as a general computer vision framework, to decode the video stream into image tensors. Server applies a pose-estimation network implemented in a machine-learning framework to each image tensor to compute joint keypoints. The pose-estimation network may be a convolutional neural network with a feature-extraction backbone and a multi-stage refinement head that outputs heatmaps for each keypoint. Server then performs triangulation or depth reconstruction, when depth information is available, to convert two-dimensional keypoints into three-dimensional joint positions. Server converts sequential joint positions into joint angles by computing relative orientations between connected segments of a skeletal structure. Server computes motion velocities as time derivatives of positions or angles, and applies smoothing filters such as low-pass finite impulse response filters or exponential moving averages. Server synchronizes these quantities with biological signals by aligning timestamps and resampling signals to a uniform time grid. The combination of joint positions, joint angles, motion velocities, and biological signals forms feature values for each time step. Server stores these feature values as rows in a time-indexed array or tensor.

Server constructs prompt sentences that describe learning and generation objectives. Server stores, for example, the following prompt sentences:

“Learn optimal screw-tightening motions from expert workers that minimize cycle time and energy use.”

“Learn high-performance but low-injury pitching motions from professional baseball players.”

“Learn lifting motions that protect the lower back while allowing heavy loads.”

“Based on the current worker motion and fatigue indicators, generate a lifting motion that minimizes lower-back stress and preserves efficiency.”

“Using the current robot arm trajectory and cycle-time statistics, generate an optimized screw-tightening motion that shortens cycle time without exceeding torque limits.” “Given this pitcher's current mechanics and mild shoulder discomfort, generate a pitching motion that maintains velocity while reducing shoulder joint load.”

“Given the user's high stress level, generate a movement sequence that relaxes the shoulders and neck while keeping the required work pace.”

Server encodes each prompt sentence into a numerical representation. In one embodiment, server uses a transformer-based language encoder. Server tokenizes the prompt sentence into subword tokens, maps tokens to embeddings, applies multiple self-attention layers, and outputs a fixed-length context vector. This context vector captures semantic information about optimization goals, constraints, and task type, and directly conditions the generative AI model.

Server implements the generative AI model as a neural network that outputs motion patterns. In one embodiment, server uses a sequence model formed by a stack of temporal encoder-decoder layers. The encoder receives a sequence of historical feature values representing recent motion and biological state, while the decoder produces a sequence of future joint angles and joint positions. Server concatenates or otherwise fuses the context vector from the prompt sentence with the encoded motion features. For example, server may apply cross-attention from the decoder to both the encoded motion sequence and the encoded prompt, allowing the decoder to generate motions that specifically reflect the described objective.

Server trains the generative AI model using stored data of expert or high-quality motions. Server uses a learning process that optimizes a loss function combining multiple terms. A reconstruction term penalizes differences between generated motion sequences and reference expert sequences. A biomechanical regularization term penalizes large joint torques or abnormal ranges of motion, computed from estimated loads based on joint angles and velocities. A smoothness term penalizes high temporal derivatives of joint angles, reducing jerkiness. A performance term encourages outcomes that satisfy performance indices, such as minimal cycle time, high speed, or required accuracy. Server uses stochastic gradient descent or an adaptive optimization algorithm to update the neural network weights. By repeating mini-batch training steps over large datasets, server converges to model parameters that can generate plausible and optimized motion patterns under different prompt conditions. Server integrates emotion and biological state into the prompt and feature representation. Server may run an emotion-recognition model, which can be another neural network analyzing facial expressions and vocal features. Server feeds output categories such as “stressed,” “fatigued,” or “excited” into a small vector that is appended to the feature values. Server also adjusts the prompt sentence to explicitly reflect the emotional state, for example, by generating prompts such as “The worker is now fatigued; generate a motion that further reduces physical load while keeping task completion acceptable.” This integration causes the generative AI model to change its output distribution based on state information without reprogramming the model architecture.

Server generates multiple candidate motion patterns for a given context. Server samples from the generative AI model with different random seeds or sampling temperatures to produce distinct candidate sequences. For each candidate, server computes evaluation indices: energy consumption, estimated from integrated mechanical power; joint load, estimated from inverse dynamics or approximate torque models; required time, measured from the predicted motion duration; and a safety index, derived from violation counts of mechanical or anatomical limits. Server ranks candidates according to a composite scoring function that may weight safety more heavily than time, or vice versa, depending on the current prompt sentence and system configuration.

Server selects an optimal motion pattern according to the evaluation indices. Server then converts the sequence of joint angles and positions into a form suitable for visualization or machine control. For visualization, server maps the sequence to a three-dimensional skeletal animation. Server uses a three-dimensional graphics engine to bind motion data to a rigged three-dimensional model. Server adjusts bone transforms for each frame, sets camera angles, and can overlay auxiliary visual elements such as trajectories, reference lines, and color-coded stress indicators. Server encodes the result as three-dimensional video data or as an animation file.

Server transmits the three-dimensional video data to terminal. Terminal decodes the video or animation and displays it on the display device. In some embodiments, terminal is a head-mounted display that overlays the generated motion onto the user's field of view, enabling augmented-reality guidance. Terminal may also display textual guidance or simplified icons to emphasize key corrections, such as bending knees more, maintaining spine alignment, or adjusting arm path.

User observes the displayed motion pattern and imitates it. User watches the three-dimensional model and adapts posture, joint motion, and timing. As user performs the optimized motion, terminal again acquires motion and biological signals and sends them to server. Server compares current motion data and current biological state data with the previously generated motion pattern. Server quantifies differences in joint angles, timing, and loads, and sequentially updates postures and trajectories in the displayed three-dimensional video so that the visualization tracks incremental improvements and provides updated correction cues. This closed feedback loop allows user to gradually converge toward a safer and higher-performance motion.

Server may also convert the optimal motion pattern into control signals for a machine apparatus or work machine. For a robot arm, server transforms joint angle sequences into joint space trajectories in the robot's coordinate system, respecting joint limits and velocity limits. Server outputs a sequence of target joint positions and velocities at discrete time steps. A robot controller receives these control signals and drives actuators accordingly. For a conveyor or assembly line machine, server may adjust timing parameters, acceleration profiles, or toolpath coordinates based on the generated motion pattern. Because the generative AI model explicitly accounts for constraints and evaluation indices, these control signals yield improved energy efficiency, reduced mechanical stress, and more consistent cycle times compared to manually tuned patterns.

This architecture improves computer technology in several ways. First, server uses a unified data structure that couples numerical feature values with prompt-conditioned context vectors within a single generative model. This eliminates the need for multiple task-specific models and reduces memory usage and model-management overhead. Second, by dynamically generating and updating prompt sentences based on biological state, emotional state, task type, and environmental conditions, server changes model behavior without recompiling or redeploying code. This reduces control-plane latency and improves adaptability of the computational pipeline.

Third, server's use of multi-objective evaluation and selection of candidate motion patterns yields more robust outputs than a single greedy generation. Because server explicitly computes energy, load, time, and safety metrics for each candidate, the system is less sensitive to outliers and can systematically trade off competing objectives. This provides improved accuracy in achieving target constraints and reduces the rate of unsafe or sub-optimal outputs compared to conventional systems that apply fixed rules after generation. Fourth, server reduces communication load and processing time by performing pre-processing at terminal and by encoding prompts as compact vectors rather than large sets of configuration parameters. Because prompt sentences express high-level user intentions, server can reuse the same neural architecture for many different scenarios while only changing text inputs. This approach reduces the complexity of configuration interfaces and facilitates efficient data management.

Fifth, server performs learning and generation using non-conventional procedures that are not mere automation of human mental steps. Server does not simply apply fixed heuristics used by coaches or workers. Instead, server uses distributed representations learned from large datasets, gradient-based optimization, and multi-objective loss functions that no human can compute manually in real time. By conditioning on prompt sentences, server discovers motion patterns that satisfy complex, sometimes non-intuitive constraints, and refines them using numerical evaluations. This combination of neural sequence modeling, prompt conditioning, and algorithmic candidate selection constitutes a specific technical solution implemented in computer hardware.

Alternative embodiments are possible. Server may implement the generative AI model as a recurrent neural network, a temporal convolutional network, or a diffusion-based generative process rather than an encoder-decoder transformer. Server may use different feature sets, such as frequency-domain representations of motion, or include contextual metadata such as equipment type. Server may use different loss functions or additional regularization terms, for example, penalizing deviation from regulatory load limits for industrial machines. Terminal may be a tablet, a stationary workstation, or a wearable device. The detection device may include advanced sensors such as depth cameras or electromyography sensors. The same general architecture and data flow apply: terminal captures motion and biological data, server processes the data, server uses prompt-conditioned generative modeling and multi-objective evaluation to produce optimized motion patterns, and terminal presents or applies the patterns in the physical world.

By these configurations, the system is not limited to abstract data analysis. The system tightly couples computer-internal data structures and algorithms with real-world acquisition hardware and actuators. The specific arrangement of a generative AI model, prompt sentences, feature extraction pipeline, and control signal generation improves the functioning of the underlying computer system in terms of adaptability, efficiency, and precision, and enables new types of real-time motion optimization and machine control that could not be practically realized by manual methods or conventional fixed-rule software.

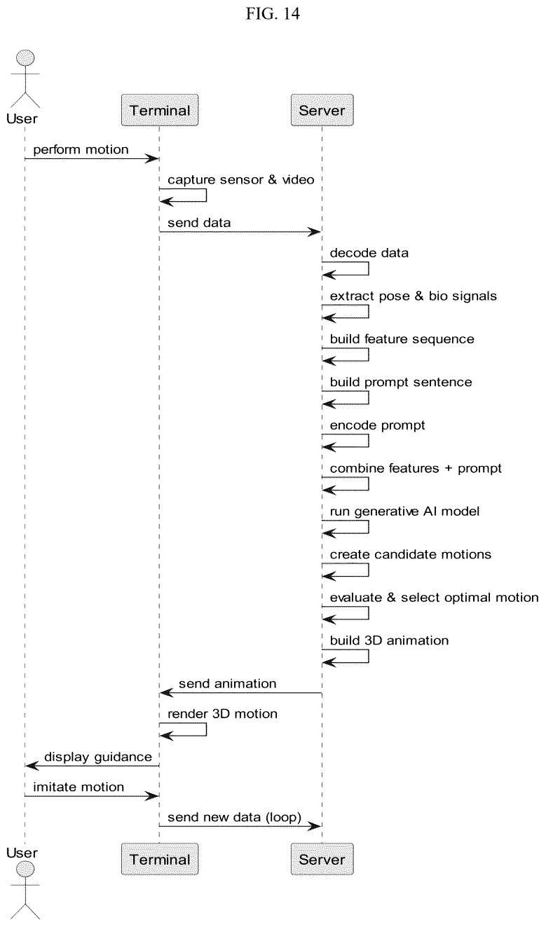

The following describes the processing flow using FIG. 14.

### Step 1:

Server initializes system resources and models.

Server loads configuration data from storage, including sensor types, sampling rates, skeletal definitions, and model hyperparameters.

Server loads pre-trained weights of the generative AI model and the language encoder into memory from a model repository.

Server allocates memory buffers for incoming time-series data (joint positions, joint angles, motion velocities, biological signals).

Input: configuration files, stored model parameters, skeletal definitions.

Processing: Server parses configuration files, instantiates neural network objects in a machine-learning framework, and binds them to hardware resources such as CPUs and GPUs.

Output: initialized generative AI model, initialized language encoder, allocated data structures ready to receive live data.

### Step 2:

Terminal acquires raw motion and biological data from the user.

Terminal activates an imaging device (for example, a camera) and one or more detection devices (for example, an inertial sensor and a heart-rate sensor).

Terminal continuously captures image frames of the user's body and sensor readings of body motion and biological state.

Input: physical movements of the user, electrical signals from sensors, light captured by the camera.

Processing: Terminal digitizes the signals, associates each frame and sensor sample with a timestamp, optionally downscales images, applies cropping, and compresses the streams.

Output: time-stamped image data and sensor data packets ready for transmission to server.

### Step 3:

Terminal transmits the captured data to the server.

Terminal opens a network connection to server using a communication protocol such as HTTP, WebSocket, or a persistent TCP socket.

Terminal sends batched image frames and sensor packets with sequence numbers to maintain ordering and detect loss.

Input: buffered image frames and sensor readings produced in Step 2.

Processing: Terminal groups data into packets, attaches metadata (timestamps, device IDs), and manages retransmission on failure.

Output: serialized data streams arriving at server's communication interface.

### Step 4:

Server receives and decodes the incoming data streams.

Server listens on the communication interface, receives packets from terminal, and reconstructs ordered sequences of images and sensor vectors.

Server decodes compressed video frames into image tensors and parses sensor packets into numerical arrays.

Input: network packets containing encoded video frames and sensor data.

Processing: Server verifies checksums, reorders packets by sequence number, decodes video formats into pixel grids, and converts sensor bytes to floating-point values.

Output: synchronized raw image sequences and raw sensor time-series stored in server memory.

### Step 5:

Server extracts three-dimensional pose and low-level features.

Server applies a pose-estimation neural network to each image frame to detect joint keypoints in image coordinates.

Server computes three-dimensional joint positions using depth information or multi-view geometry if available.

Server calculates joint angles by comparing orientations of connected bones and derives motion velocities by differentiating positions or angles over time.

Input: raw image sequences and raw sensor data from Step 4.

Processing: Server feeds image tensors into the pose model, decodes heatmaps to keypoint coordinates, computes 3D positions, calculates angles and velocity vectors, and aligns sensor streams by timestamp.

Output: time-indexed series of joint positions, joint angles, and motion velocities aligned with sensor data.

### Step 6:

Server incorporates biological signals and forms feature vectors.

Server reads biological state signals such as heart rate, respiration, or force readings from the sensor time-series.

Server resamples these signals to match the temporal resolution of the joint data.

Server concatenates joint positions, joint angles, motion velocities, and biological signals into a high-dimensional feature vector for each time step.

Input: processed joint data and raw biological signals from Step 5.

Processing: Server performs interpolation or decimation of biological signals, normalizes each channel (for example, z-score or min-max scaling), and stacks values into a fixed-length feature vector.

Output: a feature sequence representing the user's motion and state, ready for learning or generation.

### Step 7:

Server generates or selects a prompt sentence for the current task.

Server reads task metadata (for example, “lifting”, “screw-tightening”, “pitching”) and state indicators (for example, fatigue level, pain flags, emotional categories).

Server either retrieves a stored prompt template or composes a new prompt sentence using a template plus current state.

Example prompt sentences:

“Based on the current worker motion and fatigue indicators, generate a lifting motion that minimizes lower-back stress and preserves efficiency.”

“Using the current robot arm trajectory and cycle-time statistics, generate an optimized screw-tightening motion that shortens cycle time without exceeding torque limits.”

“Given this pitcher's current mechanics and mild shoulder discomfort, generate a pitching motion that maintains velocity while reducing shoulder joint load.”

Input: task type, user profile data, biological and emotional state indicators. Processing: Server applies rule-based logic or a small text generation module to assemble wording that encodes optimization goals and constraints.

Output: a prompt sentence in natural language that will condition the generative AI model.

### Step 8:

Server encodes the prompt sentence into a context vector.

Server tokenizes the prompt sentence into text tokens and maps each token to an embedding vector.

Server passes the embeddings through a language encoder comprising multiple attention layers to obtain a fixed-length context vector.

Input: prompt sentence produced in Step 7.

Processing: Server executes matrix multiplications, attention operations, and nonlinear activations within the language encoder to compute a semantic representation.

Output: a prompt-condition context vector representing the meaning of the prompt sentence.

### Step 9:

Server prepares model input by combining motion features and context.

Server selects a window of recent feature vectors representing the user's latest motion and state.

Server concatenates or otherwise fuses these feature vectors with the context vector from the prompt encoding.

Input: feature sequence from Step 6 and context vector from Step 8.

Processing: Server slices a time window, pads or truncates sequences to a fixed length, broadcasts the context vector across time steps if needed, and forms a tensor compatible with the generative AI model's input layer.

Output: a conditioned input tensor that encodes both low-level motion/state and high-level prompt intent.

### Step 10:

Server executes the generative AI model to create candidate motion patterns.

Server feeds the conditioned input tensor into the generative AI model, which may be implemented as a sequence-to-sequence neural network.

Server runs a forward pass to predict a sequence of future joint angles and positions for each candidate.

Server optionally repeats the forward pass with different sampling parameters (for example, random seeds or temperature) to produce multiple distinct candidates.

Input: conditioned input tensor from Step 9.

Processing: Server applies layer-by-layer computations (temporal convolutions, recurrence, or attention) to produce predicted sequences of joint states; stochastic sampling introduces diversity across candidates.

Output: one or more candidate motion patterns, each defined as a time-ordered list of joint angles and positions.

### Step 11:

Server evaluates each candidate motion pattern using technical indices.

Server computes energy consumption for each pattern by integrating estimated mechanical power across joints.

Server computes joint loads using approximate inverse dynamics or torque estimations from joint angles and velocities.

Server measures required time and checks for violations of joint limits or safety thresholds to derive a safety index.

Input: candidate motion patterns from Step 10 and biomechanical parameters from configuration.

Processing: Server applies numerical formulas to each time step, accumulates metrics over the sequence, and computes scalar scores for energy, load, time, and safety; server optionally forms a composite score using weighted sums.

Output: evaluation scores associated with each candidate motion pattern.

### Step 12:

Server selects an optimal motion pattern.

Server compares evaluation scores across all candidates to identify the pattern that best satisfies the prompt-specific objectives (for example, maximize safety while staying within a time limit).

Server chooses the candidate with the highest composite score or lowest cost function value.

Input: evaluation scores and candidate motion patterns from Step 11.

Processing: Server applies a selection algorithm such as argmax or argmin over the candidate set, possibly with constraints (for example, discard patterns with safety index below a threshold).

Output: a single optimal motion pattern designated for visualization or machine control.

### Step 13:

Server converts the optimal motion pattern into three-dimensional animation data.

Server maps joint angles and positions to a digital skeleton with predefined bone hierarchies.

Server computes bone transformations for each animation frame and applies them to mesh vertices associated with the skeleton.

Input: optimal motion pattern from Step 12 and skeletal/mesh data from initialization.

Processing: Server iterates over time steps, computes rotation matrices or quaternions, transforms joint coordinates into global positions, and updates vertex positions for rendering.

Output: three-dimensional animation data representing the optimal motion over time.

### Step 14:

Server encodes and transmits the three-dimensional animation to terminal.

Server encodes the animation as a video stream or an animation file (for example, a sequence of frames or an animated model format).

Server generates metadata such as duration, frame rate, and skeleton mapping.

Server sends the encoded data and metadata to terminal over the network.

Input: three-dimensional animation data from Step 13.

Processing: Server compresses the animation into a suitable format, encapsulates it with headers, and handles network transmission.

Output: animation file or stream received by terminal.

### Step 15:

Terminal renders and displays the optimized motion.

Terminal decodes the received animation or video and initializes a local rendering engine. Terminal displays the three-dimensional avatar performing the optimal motion on a screen or head-mounted display, optionally including overlays (for example, target paths, color-coded joints).

Input: encoded animation and metadata from Step 14.

Processing: Terminal converts encoded data into renderable frames, applies camera and lighting settings, and renders the frames to the display at a target frame rate.

Output: visual presentation of the optimal motion to the user in real time or near real time.

### Step 16:

User observes the guidance and attempts to imitate the motion.

User watches the three-dimensional animation and adjusts body posture, joint trajectories, and timing to match the recommended pattern.

User performs the instructed motion repeatedly to internalize the changes.

Input: visual guidance displayed by terminal in Step 15.

Processing: User interprets the visual information, executes corresponding physical movements, and may provide feedback verbally or through a user interface.

Output: updated physical motion of the user, which can be re-captured by terminal for further optimization.

### Step 17:

Server optionally converts the optimal motion pattern into machine control signals.

Server transforms the joint angles of the optimal pattern into device-specific coordinates for a machine apparatus or work machine, such as a robot arm.

Server generates a time-stamped sequence of control commands specifying target positions, velocities, and accelerations for each actuated joint or axis.

Input: optimal motion pattern from Step 12 and machine kinematic/actuator specifications. Processing: Server applies kinematic mappings, scales timing to machine capabilities, clamps values to hardware limits, and formats commands according to the machine control protocol.

Output: machine-readable control signals ready to be sent to a controller of the machine apparatus.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

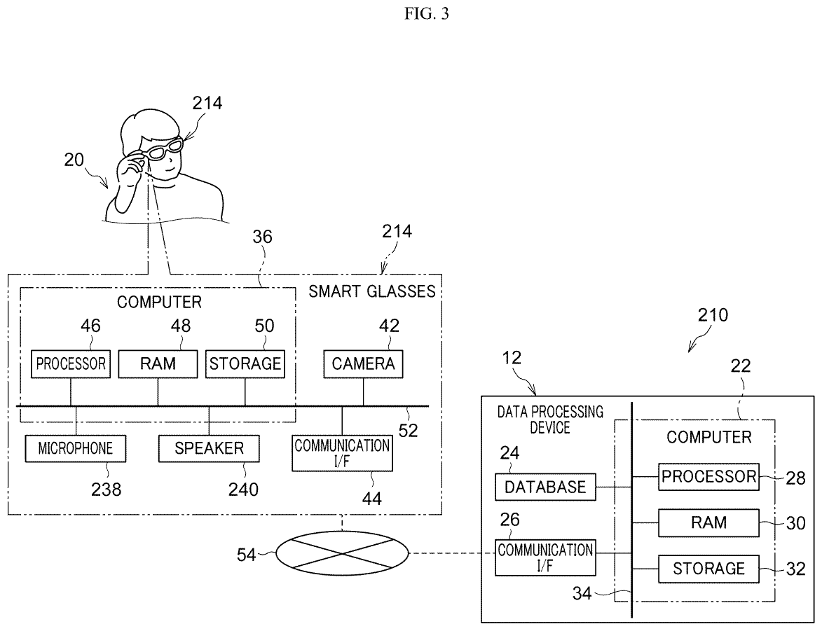

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

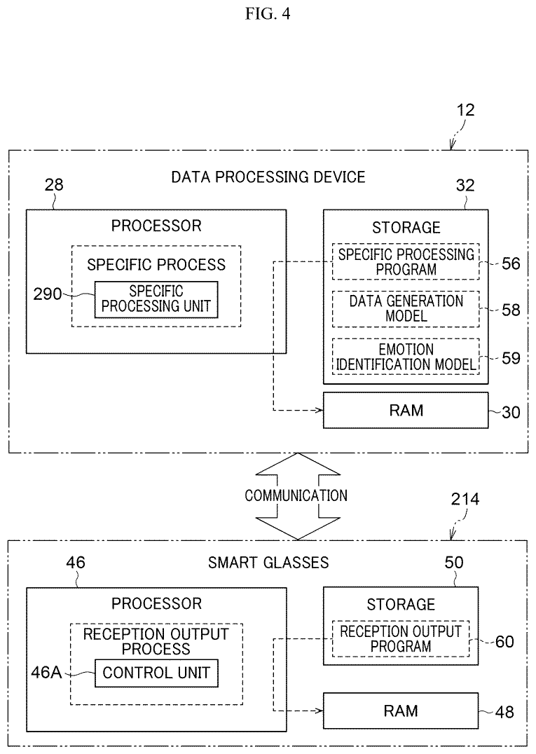

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

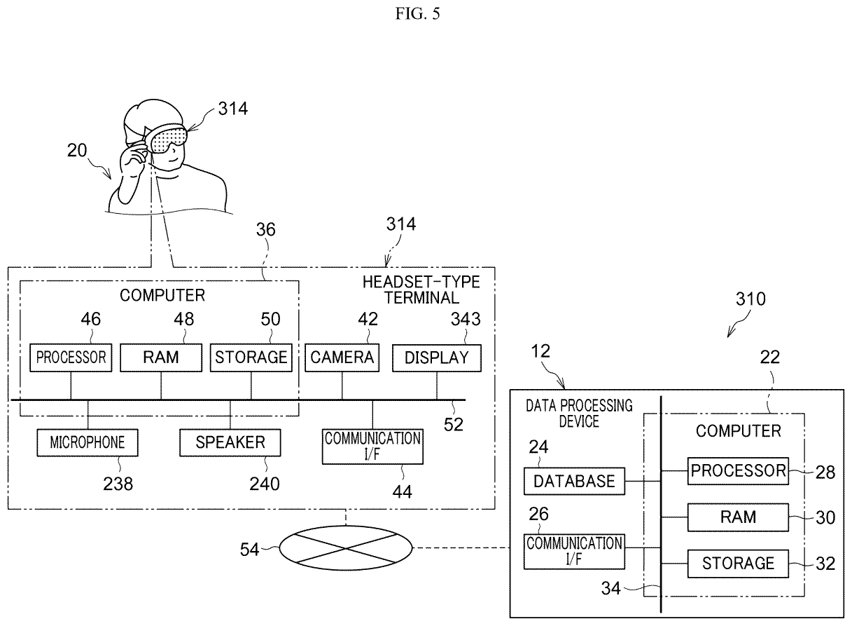

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

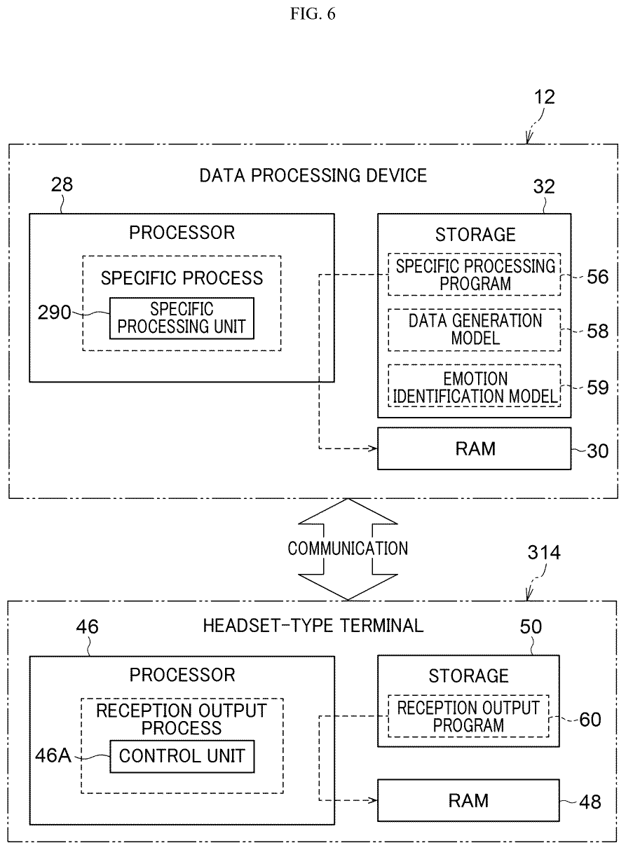

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

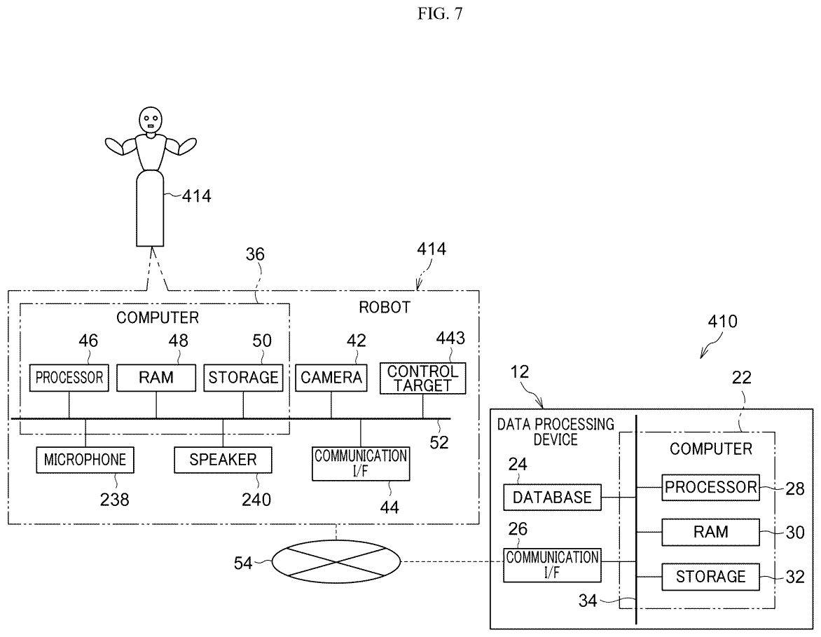

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

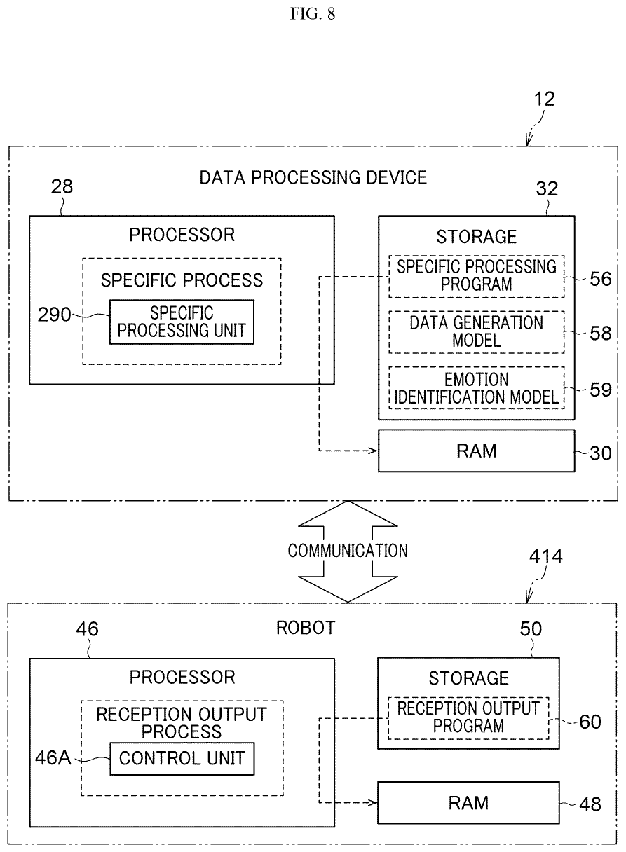

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

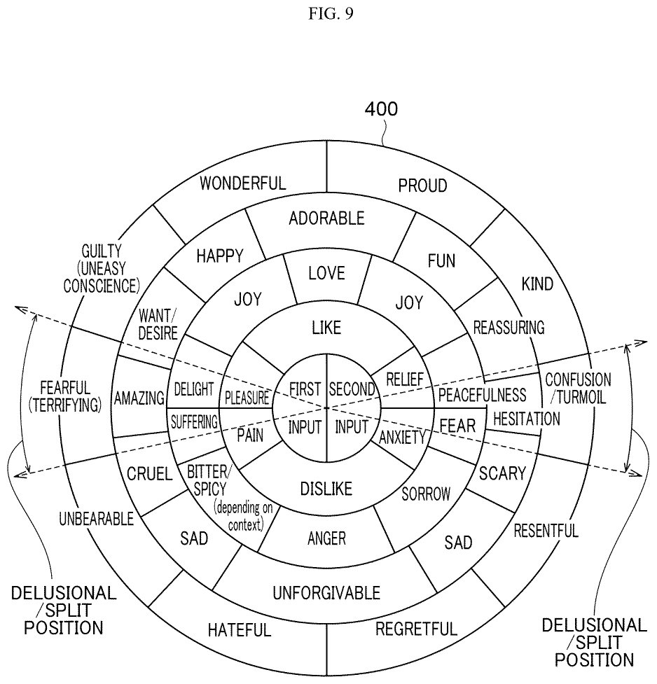

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

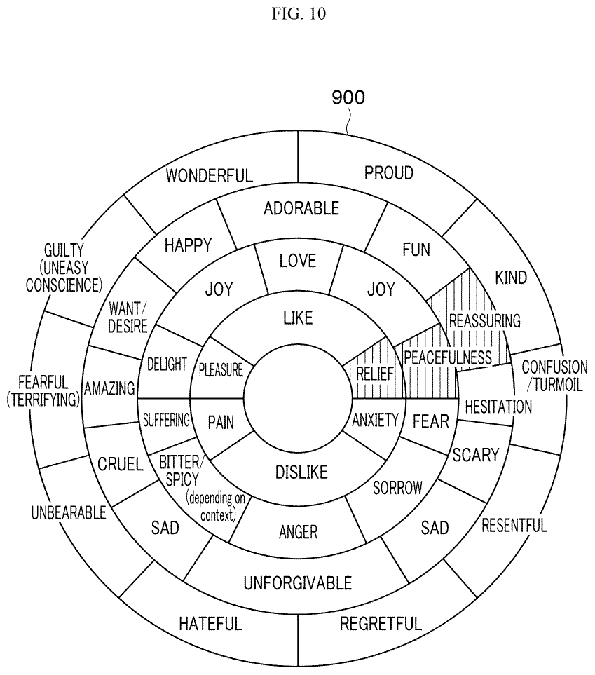

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (SaaS).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above. All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### (Supplementary 1)

A system comprising a processor,
wherein the processor is configured to
generate, for a generative AI model that is a statistical learning information processing model, a prompt sentence describing that body capability information and motion information during physical activity are to be used as training data, and input the prompt sentence and the training data into the generative AI model to cause the generative AI model to execute a learning process,
acquire, from a measurement apparatus including at least one wearable measurement device or imaging device, time-series data including biosignals and posture information of a subject during physical activity, correct the time-series data to have a unified sampling period, remove noise components from the time-series data, segment the time-series data into a plurality of motion intervals, and convert, for each motion interval, the time-series data into a feature sequence including joint angles, angular velocities, load estimation values, and heart rate values,
read, from an external information storage apparatus, a health check result of the subject, and add to the feature sequence a health status indicator including at least a maximum allowable heart rate, a joint function limitation, and past injury information,
execute, by using an analysis information processing model, an inference process that takes the feature sequence and the health status indicator as inputs, simultaneously estimates a motion capability index of the subject and a joint load index of the subject, and outputs a motion pattern that increases capability expression while suppressing injury risk,
generate, on the basis of an estimation result obtained from the analysis information processing model, a generation instruction prompt sentence described in a natural language and including at least attributes of the subject, a type of physical activity, motion-related problems, and an output format, input the generation instruction prompt sentence into the generative AI model, and cause the generative AI model to execute a generation process to output digital data including a sequence of joint angles and time information representing an optimal motion pattern in which both a capability index and an injury risk index are taken into account,
associate the digital data representing the optimal motion pattern with a three-dimensional skeletal model, convert the digital data into continuous three-dimensional motion data by using inverse kinematics processing and interpolation processing, and generate video data in which measured motion data of the subject and the optimal motion pattern are displayable for comparison, and
transmit the video data to a terminal device and enable a display device of the terminal device to visually present the measured motion of the subject and the optimal motion pattern simultaneously from at least one viewpoint.

### (Supplementary 2)

The system according to supplementary 1,
wherein the processor is configured to
read, from an information storage apparatus, a reference data set including motion data during physical activity of a plurality of skilled performers as reference motion data, and generate the prompt sentence such that the reference data set is used as teacher data in the statistical learning process of the generative AI model and in a learning process of the analysis information processing model.

### (Supplementary 3)

The system according to supplementary 1,
wherein the processor is configured to
associate the three-dimensional motion data representing the optimal motion pattern with the measured motion data of the subject, display the measured motion data and the optimal motion pattern in parallel within the video data, and superimpose annotation information that emphasizes differences between the measured motion data and the optimal motion pattern so that the subject can maximally express physical capability.

### Application Example 1

### (Supplementary 1)

A system comprising a processor,
wherein the processor is configured to
acquire biological information and motion information of a user from an information acquisition apparatus including at least one sensor or imaging apparatus, and generate body capability information and motion information as time-series data,
generate a prompt sentence that instructs a generative AI model to perform a learning process using the body capability information and the motion information, and cause the generative AI model to execute the learning process based on the prompt sentence,
acquire health state information of the user and motion information of the user during a task from the information acquisition apparatus,
generate a prompt sentence that instructs the generative AI model to generate an optimal motion pattern in which the user exerts a maximum capability in a predetermined task while reducing a risk of fatigue and injury, the prompt sentence being based on the health state information of the user and the motion information of the user during the task, and cause the generative AI model to execute a generation process of the optimal motion pattern based on the prompt sentence, convert the optimal motion pattern output from the generative AI model into numerical data including a sequence of joint angles and a sequence of body-part trajectories,
generate three-dimensional motion image data by applying the numerical data to a three-dimensional display object having a skeletal structure and a surface shape in a virtual space,
transmit the three-dimensional motion image data to a portable display apparatus via a communication line,
control the portable display apparatus to display the three-dimensional motion image data in a manner superimposed on a real space so that the user is able to compare, in real time, an own motion with the optimal motion pattern,
acquire updated motion information of the user, who has corrected the own motion based on the display, from the information acquisition apparatus, and repeatedly execute generation of the optimal motion pattern and generation of the three-dimensional motion image data as a feedback process, and
generate a prompt sentence that instructs the generative AI model to generate a motion guidance text for the user, the prompt sentence being based on at least one of the body capability information, the health state information, task content information, and the optimal motion pattern, obtain the motion guidance text generated by the generative AI model, and
transmit the motion guidance text to the portable display apparatus.

### (Supplementary 2)

The system according to supplementary 1,
wherein the processor is configured to include, in the prompt sentence for the learning process to the generative AI model, information instructing that motion information of a plurality of highly skilled workers is to be used as reference data.

### (Supplementary 3)

The system according to supplementary 1,
wherein the processor is configured, in generating the three-dimensional motion image data, to perform at least one of temporal interpolation processing and smoothing processing on the optimal motion pattern generated by the generative AI model, and to construct a continuous three-dimensional motion trajectory of the three-dimensional display object so that the user is able to safely exert a maximum capability.

### Example 2

### (Supplementary 1)

A system comprising a processor,
wherein the processor is configured to
acquire, for a subject, time-series image information and biological signal information from an imaging device and a biological measurement device, respectively, and to associate the time-series image information and the biological signal information with measurement time information and subject attribute information to generate analysis data,
extract, by executing posture estimation processing on the time-series image information included in the analysis data, time-series posture information indicating positions of body parts of the subject, compute feature information including at least joint angle information, movement speed information, motion phase information, and biological response information based on the time-series posture information and the biological signal information, and derive capability index information and load index information by comparing the feature information with reference feature information of professional exercise performers,
input the feature information, the capability index information, and the load index information into a machine learning model that has been trained in advance on reference data, and estimate, by using the machine learning model, performance index information, injury risk index information, and motion classification information for the subject, structure analysis result information including at least the subject attribute information, the feature information, the capability index information, the load index information, and an estimation result of the machine learning model into a natural-language description, generate a prompt sentence including an instruction statement according to a content of the analysis result information and a training objective of the subject, and perform generation processing of instructing a generative AI model, by the prompt sentence, to generate subject-specific training content and motion improvement information, and
output the training content and the motion improvement information received from the generative AI model as advice information associated with numerical index information, and, when necessary, associate and present the advice information with a three-dimensional motion model or visual display information.

### (Supplementary 2)

The system according to supplementary 1,
wherein the processor is configured to use, for training the machine learning model, reference feature information based on motion data and biological signal data of a plurality of professional exercise performers as teacher data, and to generate the reference feature information used for the comparison, the capability index information, and the load index information.

### (Supplementary 3)

The system according to supplementary 1,
wherein the processor is configured to construct the three-dimensional motion model or the visual display information based on a motion pattern presented by the generative AI model and the posture information, and to emphasize and display recommended joint angle information, body center-of-gravity movement information, and landing motion information so that the subject can exhibit maximum physical capability while reducing injury risk.

### Application Example 2

### (Supplementary 1)

A system comprising a processor,
wherein the processor is configured to
perform a learning process on time-series data by using a prompt sentence that instructs a generative AI model to learn attributes related to a body and information related to physical motion,
acquire, by using a detection device and an imaging device, biological state information and physical motion information of a target subject, and extract, from the acquired information, feature values including joint positions, joint angles, motion velocities, and biological signals,
input the extracted feature values and the prompt sentence to the generative AI model and execute a generation process in which the generative AI model generates a motion pattern that allows the target subject to satisfy a predetermined performance index while reducing load and damage risk,
generate three-dimensional video data that visualizes the motion pattern by deforming three-dimensional shape data having a skeletal structure along a time axis on the basis of the motion pattern generated by the generative AI model,
transmit the three-dimensional video data to an information presentation device and present the three-dimensional video data as instruction information for improvement of motion of the target subject or of a machine apparatus,
dynamically generate or update the prompt sentence to be input to the generative AI model in accordance with at least one of a biological state, an emotional state, a task type, an environmental condition, and an evaluation index related to the target subject, and
cause the generative AI model to generate a plurality of candidate motion patterns, evaluate the plurality of candidate motion patterns on the basis of at least one of energy consumption, joint load, required time, and a safety index, select an optimal motion pattern from among the plurality of candidate motion patterns, and convert the optimal motion pattern into a control signal for a machine apparatus or a work machine so as to adjust operation of the machine apparatus or the work machine.

### (Supplementary 2)

The system according to supplementary 1,
wherein the processor is configured to perform the learning process for the generative AI model by using a prompt sentence that instructs the generative AI model to read reference data including at least one of motion data of a subject performing specialized physical motions and work motion data of a skilled worker.

### (Supplementary 3)

The system according to supplementary 1,
wherein the processor is configured to compare the motion pattern generated by the generative AI model with current motion data and current biological state data of the target subject, and, based on a result of the comparison, sequentially update postures and motion trajectories in the three-dimensional video data so that the target subject can exert ability to a maximum extent while suppressing damage risk.

What is claimed is:

1. A system comprising:
circuitry configured to:
acquire, via a communication interface coupled to a packet-switched network, time-series data comprising biosignals and posture information from a measurement apparatus including at least one wearable measurement device or imaging device, correct the time-series data to a unified sampling period, remove noise components from the time-series data, segment the time-series data into a plurality of motion intervals, and convert, for each motion interval, the time-series data into a feature sequence comprising joint angles, angular velocities, load estimation values, and heart rate values;
read, from an external information storage apparatus accessed via the communication interface, a health check result of a subject, and add to the feature sequence a health status indicator comprising a maximum allowable heart rate, a joint function limitation, and past condition information;
execute, using an analysis information processing model, an inference process that takes the feature sequence and the health status indicator as inputs, simultaneously estimates a capability index and a load index for the subject, and outputs a motion pattern that increases capability expression while suppressing load risk;
generate, based on an estimation result from the analysis information processing model, a generation instruction prompt sentence in a natural language comprising subject attributes, a type of physical activity, motion-related parameters, and an output format, transmit the generation instruction prompt sentence to a generative neural network model via the communication interface, and obtain digital data comprising a sequence of joint angles and time information representing an optimal motion pattern; and
associate the digital data with a three-dimensional skeletal model, convert the digital data into continuous three-dimensional motion data using inverse kinematics processing and interpolation processing, generate video data in which measured motion data of the subject and the optimal motion pattern are displayable for comparison, and transmit the video data to a terminal device via the communication interface.

2. The system according to claim 1, wherein the circuitry is configured to generate, for the generative neural network model, a prompt sentence describing that body capability information and motion information during physical activity are to be used as training data, and input the prompt sentence and the training data into the generative neural network model to cause the generative neural network model to execute a learning process prior to the generation of the optimal motion pattern.

3. The system according to claim 2, wherein the circuitry is configured to read, from the external information storage apparatus, reference motion data associated with a high-performance reference population, and to include the reference motion data as training data in the prompt sentence for the learning process.

4. The system according to claim 2, wherein the circuitry is configured to apply a data augmentation function to the training data by generating synthetic feature sequences via perturbation of the joint angles and angular velocities within constraint bounds derived from the health status indicator, and to include the augmented training data in the learning process.

5. The system according to claim 1, wherein the circuitry is configured to enable a display device of the terminal device to visually present the measured motion data of the subject and the optimal motion pattern simultaneously from at least one viewpoint selectable by a user.

6. The system according to claim 5, wherein the circuitry is configured to receive a viewpoint selection instruction from the terminal device via the communication interface, apply a camera projection function to the three-dimensional motion data to render the video data from the selected viewpoint, and retransmit the rendered video data to the terminal device.

7. The system according to claim 5, wherein the circuitry is configured to compute a deviation metric between the measured motion data of the subject and the optimal motion pattern for each motion interval, overlay the deviation metric as a color-coded visualization on the video data, and transmit the annotated video data to the terminal device.

8. The system according to claim 1, wherein the circuitry is configured to apply a multi-modal synchronization function to the time-series data by aligning timestamps across biosignal channels and posture information channels using a reference clock signal, prior to segmenting the time-series data into motion intervals.

9. The system according to claim 8, wherein the circuitry is configured to apply a signal quality assessment function to each channel of the time-series data, flag channels with signal quality below a threshold, and apply a channel-specific noise removal filter to flagged channels prior to the conversion into the feature sequence.

10. The system according to claim 1, wherein the circuitry is configured to store the feature sequence, the health status indicator, and the digital data representing the optimal motion pattern in a storage device in association with a subject identifier and a session timestamp, and to retrieve stored data in response to a historical comparison request from the terminal device.

11. The system according to claim 10, wherein the circuitry is configured to apply a temporal comparison function to feature sequences stored across a plurality of sessions to compute a trend metric for the capability index and the load index, and to include the trend metric in a progress report transmitted to the terminal device.

12. The system according to claim 10, wherein the circuitry is configured to detect a threshold-crossing event when the capability index or the load index stored across consecutive sessions shows a monotone progression exceeding a configurable threshold, and to transmit a notification to the terminal device via the communication interface upon detecting the event.

13. The system according to claim 1, wherein the circuitry is configured to apply an inverse kinematics validation function to the continuous three-dimensional motion data to detect joint angle configurations that exceed anatomical range-of-motion limits derived from the health status indicator, and to apply a correction function to the digital data to bring the configurations within the limits prior to generating the video data.

14. The system according to claim 13, wherein the circuitry is configured to log each detected range-of-motion violation and the corresponding correction applied in a storage device as a motion safety record, and to include a summary of the motion safety record in the video data transmitted to the terminal device.

15. The system according to claim 1, wherein the circuitry is configured to apply a segmentation quality function to the plurality of motion intervals to assign a quality score to each interval based on signal completeness and noise level, and to exclude intervals with quality scores below a threshold from the inference process executed by the analysis information processing model.

16. The system according to claim 1, wherein the circuitry is configured to receive a motion parameter adjustment instruction from the terminal device via the communication interface, regenerate the generation instruction prompt sentence incorporating the adjusted parameters, retransmit the regenerated prompt sentence to the generative neural network model, and generate updated video data based on the updated digital data.

17. The system according to claim 16, wherein the circuitry is configured to store the adjusted motion parameters and the associated updated digital data in a storage device as a parameter history record, and to provide the parameter history record to the terminal device to enable comparison of motion patterns across multiple adjustment iterations.

18. A system comprising:
circuitry configured to:
acquire, via a communication interface coupled to a packet-switched network, time-series data comprising biosignals and posture information from a measurement apparatus, segment the time-series data into a plurality of motion intervals after correcting to a unified sampling period and removing noise, and convert each motion interval into a feature sequence comprising joint angles, angular velocities, load estimation values, and heart rate values;
read a health check result from an external information storage apparatus accessed via the communication interface, and add a health status indicator comprising a maximum allowable heart rate, a joint function limitation, and past condition information to the feature sequence;
execute an inference process using an analysis information processing model that takes the feature sequence and the health status indicator as inputs to simultaneously estimate a capability index and a load index, and generate a generation instruction prompt sentence in a natural language based on the estimation result comprising subject attributes, a type of physical activity, motion-related parameters, and an output format;
transmit the generation instruction prompt sentence to a generative neural network model via the communication interface to obtain digital data comprising a sequence of joint angles and time information representing an optimal motion pattern; and
associate the digital data with a three-dimensional skeletal model, convert the digital data into continuous three-dimensional motion data using inverse kinematics processing and interpolation processing, generate video data in which measured motion data of the subject and the optimal motion pattern are displayable for comparison, and transmit the video data to a terminal device via the communication interface.

19. The system according to claim 18, wherein the circuitry is configured to apply an inverse kinematics validation function to the continuous three-dimensional motion data to detect joint angle configurations exceeding anatomical range-of-motion limits derived from the health status indicator, and to apply a correction function to bring the configurations within the limits prior to generating the video data.

20. A method comprising:
acquiring, via a communication interface coupled to a packet-switched network, time-series data comprising biosignals and posture information from a measurement apparatus, correcting the time-series data to a unified sampling period, removing noise components, segmenting the time-series data into a plurality of motion intervals, and converting, for each motion interval, the time-series data into a feature sequence comprising joint angles, angular velocities, load estimation values, and heart rate values;
reading, from an external information storage apparatus, a health check result of a subject, and adding to the feature sequence a health status indicator comprising a maximum allowable heart rate, a joint function limitation, and past condition information;
executing, using an analysis information processing model, an inference process that takes the feature sequence and the health status indicator as inputs, simultaneously estimating a capability index and a load index, and outputting a motion pattern that increases capability expression while suppressing load risk;
generating, based on an estimation result from the analysis information processing model, a generation instruction prompt sentence in a natural language comprising subject attributes, a type of physical activity, motion-related parameters, and an output format, transmitting the generation instruction prompt sentence to a generative neural network model via the communication interface, and obtaining digital data comprising a sequence of joint angles and time information representing an optimal motion pattern; and
associating the digital data with a three-dimensional skeletal model, converting the digital data into continuous three-dimensional motion data using inverse kinematics processing and interpolation processing, generating video data in which measured motion data of the subject and the optimal motion pattern are displayable for comparison, and transmitting the video data to a terminal device via the communication interface.
